(19) United States
(12) Patent Application Publication
(10) Pub. No.: US 2024/0331822 A1
(43) Pub. Date: Oct. 3, 2024

(54) SYSTEM AND METHOD FOR CREATING MEDICAL DOCUMENTS

(71) Applicant: Medstreme, LLC, Safety Harbor, FL (US)

(72) Inventors: N.R. Chandrasekar, Canton, MA (US); Thomas Osypka, Safety Harbor, FL (US)

(73) Assignee: Medstreme, LLC, Safety Harbor, FL (US)

(21) Appl. No.: 18/620,539

(22) Filed: Mar. 28, 2024

Related U.S. Application Data
(60) Provisional application No. 63/455,173, filed on Mar. 28, 2023.

(51) Int. Cl. G16H 15/00; G16H 40/67

(52) U.S. Cl. CPC G16H 15/00; G16H 40/67

(57) ABSTRACT

A system automatically creates medical documents, such as post-operative documents via a plurality of touch screen units. Generated on a GUI of a touch screen unit is a user interactive animated visual representation of at least a portion of a human body representative of the patient including a plurality of selectable anatomical anomalies that may be relevant to the patient. Once one or more portions of the human body portions are selected, one or more medical procedures that may be associated with respect to the selected human body portions are displayed on the GUI. A user then selects, via the GUI, one or more medical procedures performed on the patient with respect to the selected human body portions. A surgical report is then automatically generated for the one or more selected performed medical procedures. One or more Artificial Intelligence (AI) techniques are utilized for determining the one or more medical procedures identified on the GUI and for generating the medical report.

### CROSS REFERENCE TO RELATED APPLICATIONS

This application claims priority to U.S. Patent Application Ser. No. 63/455,173 filed Mar. 28, 2023, which is incorporated herein by reference in its entirety.

### BACKGROUND

### Field

The disclosed embodiments generally relates to medical documentation systems, and more specifically to a system and method for automatically generating one or more medical documents, and more particularly, to generating medical documents utilizing hierarchically-organized database views, and automated methods for generating medical reports.

### Description of Related Art

In the medical profession, generating bills for services performed on a patient is time consuming. For example, in a typical scenario, a surgeon may perform a procedure on a patient. After the procedure, the surgeon will dictate operation notes or generate handwritten notes regarding what procedures were performed and give these notes to his/her staff. The staff will then attempt to generate billing codes associated with each procedure that was performed. These billing codes may correspond to approved billing codes used by an insurance provider.

Generating bills in this manner is not only costly with respect to the time spent generating a bill, but prone to errors due to multiple parties being involved in generating the bill. For example, the doctor's notes may be poorly organized or presented, may include non-standard medical terminology, and/or may be partially provided in a language other than the primary language of the staff person generating the bill. Still another problem associated with generating bills in this manner is that the doctor may be unable to recall all of the procedures that were performed on the patient. For instance, numerous documents are often created in connection with the delivery of medical services to patients. For example, at the conclusion of a surgical procedure, a surgeon typically creates a number of documents relating to the procedure. One of these documents is known as an operative report or operative note. Generally speaking, an operative report is intended to contain sufficient information to identify the patient, support the diagnosis, justify the treatment, document the description of the procedures, methodologies, and promote continuity of care. To this end, an operative report typically includes the name of the patient, the date and location of the surgery, the names of the primary surgeon and his or her assistants, findings, the procedures used during the surgery, an identification of any specimens removed from the patient, estimated blood loss, a description of any complication that may have occurred, a postoperative diagnosis and course, and the patient's discharge condition. The operative report becomes part of the patient's medical record.

Currently, most operative reports are created through dictation and transcription. That is, upon completion of a procedure, the surgeon uses a phone to dial a pre-assigned number and dictates the operative report over the phone. The report is then transcribed, usually at a remote location taking varying amounts of time. The transcribed report is made available to the surgeon for review, editing, and final sign off. The transcribed report may be returned to the surgeon in hard-copy or electronic format, e.g., by electronic mail (email) or an electronic health records (EHR) system.

There are a number of problems that arise with the dictation of operative reports. First, the process of dictation, transcription and review can take days or even weeks to complete. Such delays can impact the hospital because an approved operative report may be needed before a hospital can bill for a procedure. Delays in the creation of such reports thus delay payments to hospitals. In addition, information in the report may be needed by the next caregiver. Accordingly, surgeons are under pressure to complete and sign operative reports as soon as possible.

As an alternative to dictation, computer-based, form systems have been created for generating operative reports for a limited number of cases. With these systems, a computer program includes a plurality of forms. One or more of the forms may be selected by a surgeon accessing the system through a computer terminal. The system may be locally stored on the computer terminal, or it may be remotely located, such as through a web server.

These systems, however, have not gained widespread adoption or use in the medical field. In fact, some of the systems are seen as being more difficult and time consuming than the dictation systems that they are intended to replace. In addition, such systems, at most, merely create an operative report. Thus, one of the most challenging problems a software developer faces when designing a database system is creating a data entry mechanism that allows users to efficiently record information. In many environments, users operate under significant time pressures and are unwilling or unable to spend time on laborious data entry procedures. In order for a data entry mechanism to be effective, it must be fast, complete, and reconfigurable. In many cases, the data entry mechanism must also map onto small display screens or onto limited space within larger screens.

Currently, most existing database systems use a forms-based data entry mechanism, which fail to satisfy the requirements listed above since most databases include a large number of fields (categories) and elements. Since the number of fields that can be displayed on a form at any one time is very small, a user must navigate through multiple complex forms in order to enter data. In addition, forms-based systems are difficult, if not impossible, to reconfigure without programming.

Some existing database systems use data navigation mechanisms that are based on hierarchically organized representations of the data. One set of prior art techniques for navigating through hierarchically organized database views is based on a diagrammatic representation of the hierarchy as a whole. In these techniques, a user moves around the hierarchy by selecting nodes on the diagram using a mouse or other pointing device. Since the hierarchy is very large, only those nodes that lie near the last node selected are displayed. The user can manually move the viewing window around (using scroll bars, for instance) and can reveal or hide levels of the hierarchy diagram by manually opening or closing nodes.

These techniques are designed to allow a user to view data elements in the context of the overall structure of the hierarchy and to visualize the logical relationships between data elements. However, the emphasis on overall structure makes these approaches ill-suited to the task of data entry. As the user moves down the hierarchy, he sees not only the nodes that represent possible choices for the next selection, but also large amounts of information that are irrelevant to the current data being entered. In addition, because much of the hierarchy diagram must, by necessity, be off-screen at any point in time, it is often difficult for the user to ascertain how he has reached a particular point in the hierarchy or how the displayed information fits within the overall structure.

Also, present navigational techniques for hierarchical file directory structures that display the names of the files in a selected directory along with the path to that directory are limited to file selection, and do not address the entry or review of database information. Therefore, there remains a need for an easy-to-use interface, for entry, update, and review of data from a hierarchically-organized database view.

There is a particular need for such a system in the creation and management of medical records and the generation of reports from these records. For example, currently many medical reports are generated from transcription of a physician-dictated report. This procedure is inefficient and costly since the process requires manual and inaccurate transcription. Furthermore, such a procedure is time-consuming to a physician, who must review and edit the transcribed report.

Attempts at solving this problem have focused on computer-based form systems. In these systems, a user enters information into a series of forms, to populate a database. These form-based systems have fundamental drawbacks. First, the systems are not flexible. Therefore, users cannot easily tailor the forms to their preferences. This poses serious issues in medical reporting, where physicians and medical institutions have specific preferences for their medical reports. Second, completing the forms is time consuming, as a user must go through and complete entries in many fields in the form. Therefore, there remains a need for an efficient, flexible, user-friendly interface for recording medical information and creating reports from the recorded information. Finally, current medical records management systems do not provide an effective interface for formulating queries on recorded clinical data and generating reports from this data. Such a feature is important to physicians for medical accreditation purposes as well as for reviewing clinical data for scientific study. At best, existing query tools use some flavor of Query-By-Example (QBE) to form SQL queries on the underlying database. The principal failing of this approach when applied to the medical domain is that it forces the user to formulate a query in terms of the relationships that exist between data items in the database rather than in terms of the clinical relationships that naturally exist between the data items. Therefore, there remains a need for a query generation tool for medical data, which allows a query to be formed in an intuitive manner by taking advantage of the clinical relationships between data items, both to assist the user in locating data items and to express the relationship between these data items within the query itself.

Thus, there exists a need to provide an easy to use and flexible interface for the entry of medical information into a database, and generation of customized reports from that information.

### SUMMARY

The illustrated embodiments relate to a system and method for automatically creating medical documents, such as documents pertaining to a surgical procedure, in a quick and efficient manner. A surgeon or health care provider is able to generate more accurate and relevant medical documents, such as operative reports, post-operative orders, electronic and paper prescriptions, and post-operative discharge instructions, that include appropriate and correct codes, such as Current Procedural Terminology (CPT) and International Statistical Classification of Diseases and Related Health Problems (ICD) codes for billing purposes. The inventive system and method may include one or more easy to use Graphical User Interfaces (GUIs) presented on a touchscreen unit that allows the surgeon or health care provider to create and approve, e.g., electronically sign, one or more medical documents immediately upon completing a procedure, examination, or surgery.

A medical document management system includes a document server that, in turn, includes a plurality of modules. Specifically, the document server includes a scheduling entity, a template builder, a document constructor, a document distribution entity, a data communication facility, and a human-machine interface (HMI) entity. In addition, the system may include a scheduling database, a template database, and one or more database management systems that interface to the scheduling and template databases.

A plurality of document forms, such as form operative reports, form recovery room instructions, and form discharge instructions, may be created and loaded into the template database. In an embodiment, the form documents are predefined by a system developer for a wide range of surgical procedures. Each form document may correspond to particular surgical procedure, and may contain generally or commonly accepted information concerning that procedure. Included in the form documents may be one or more placeholders or blanks that mark locations where patient or procedure specific information is to be inserted.

A hospital administrator may create a schedule of surgical procedures that are to be performed in one or more of the hospital's operating rooms, and this schedule may be accessed by the system. For each procedure, the administrator may enter the patient's name, date of birth, Medical Record Number (MRN), date and time of surgery, type of surgical procedure, operating room, name of surgeon, name of anesthesiologist, etc. In an embodiment, the administrator may upload the schedule to the system. The database management system may store the scheduling information in the scheduling database.

The system also may include a plurality of touchscreen units that are located in the operating rooms and/or other selected locations, and are coupled to the server. In an embodiment, the touchscreen units may or may not include a separate keyboard, keypad, mouse or other pointing device, such as a stylus. After completing a surgical procedure, the surgeon may utilize the touchscreen unit in the operating room to initiate the creation of one or more medical documents, such as an operative report. Specifically, the surgeon may log onto the document server using a predefined username and password. Upon a successful login, the scheduling entity may search the schedule database for the surgical procedures scheduled for that day. The HMI entity in cooperation with the scheduling entity may present the list of procedures on the touchscreen unit. The list may be limited by operating room, or may include only the procedures scheduled for the logged in surgeon. Each entry in the list may include patient name, related demographic data, type of surgery, and time of surgery.

The surgeon may select the just-completed procedure from the list of procedures using, for example, a touch gesture. In response, the document constructor may access the template database, and retrieve the one or more form documents for the type of procedure selected by the surgeon. The HMI entity may present one or more Graphical User Interfaces (GUIs) on the touchscreen unit prompting the surgeon to enter information for completing the one or more medical documents, such as estimated blood loss, the skin closure used, the occurrence of any complications, the pain and/or other medications to be prescribed. The document constructor may also retrieve patient-specific information for the patient on whom the procedure was performed. Utilizing the information or data entered by the surgeon, and the retrieved patient-specific information, the document constructor may automatically create completed versions of the one or more medical documents. For example, the document constructor may automatically generate an operative report, a set of recovery room instructions, a set of discharge instructions, and one or more prescriptions for the medication specified by the surgeon. The patient-specific and surgery-specific information received by the document server may be inserted into the forms at the locations defined by the placeholders or blanks.

The document constructor may generate these documents in completed form without any further input from the surgeon other than the selection of the just completed procedure, and the entry of the prompted data or information regarding the procedure. For example, the operative report, as generated by the document constructor, may include patient name, date of birth, sex, pre-operative diagnosis, post-operative diagnosis, date of surgical procedure, Current Procedural Terminology (CPT) code, a description of the surgical procedure as performed, a description of any complication that occurred, etc. Appropriate order sets, recovery room instructions, discharge instructions, and prescriptions may similarly be generated in an automatic manner by the document server.

The surgeon may accept the auto-generated, completed documents by applying his or her electronic signature (e-signature) to the documents. The document distribution entity may transmit the signed, auto-generated documents to one or more predetermined locations or destinations. For example, the operative report and recovery room instructions may be sent to the hospital's patient record system, while the prescription may be sent to a retail or in-house pharmacy. In addition, the document distribution entity may print hard copies of one or more of the auto-generated documents, such as recovery room instructions. The hard copies may be added to the patient's physical file, and transported with the patient as he or she is moved from the operating room to the recovery room. Nonetheless, the surgeon may, if necessary, edit, modify or revise the auto-generated operative report or other documents.

In an embodiment, the surgeon may access the document server from other devices besides the touchscreen units located in the operating rooms. For example, the surgeon may use a mobile device, a desktop device, or other computing platform to access the document server, and initiate the generation of the completed documents.

As described, post-operative documents are automatically created by the system without the surgeon having to dictate any material, manually enter data through typing, or wait for transcription to be performed. In most instances, all of the required medical documents can be created automatically with just a few touches on the touchscreen unit by the surgeon. In addition, the documents are created without the surgeon having to operate a keyboard, a pointing device, or a paper and pen.

In an embodiment, the HMI entity in cooperation with the scheduling entity may provide an operating room dashboard. The dashboard may present a list of all of the procedures being performed in the hospital's operating rooms and/or it may present all of the procedures of a surgeon on the current day. The dashboard may be presented on a display unit located in a surgeon's lounge or other desired location. The dashboard may include information indicating when a procedure started, and when it was finished, and this information may be continuously updated. By reviewing the information presented on the dashboard, a surgeon can quickly determine whether a particular procedure is running behind schedule, thus delaying another procedure that the surgeon is scheduled to perform in that same operating room. The operating room dashboard may also be accessed from other devices, such as mobile and desktop devices.

In an embodiment, surgeons may specify one or more default conditions for use in the generation of medical documents. By specifying one or more default conditions, a surgeon may direct the system to automatically generate medical documents tailored to the surgeon's particular practice. The one or more default conditions may include an identification of the set of documents to be created following a particular procedure, the surgical code-(Current Procedural Terminology-CPT) identifying the procedure, a preferred medication, a preferred dosage, etc. A surgeon also may revise the form documents and/or create customized document templates that more closely follow his or her practice. For example, an orthopedic surgeon may create an individualized operative report template, recovery room instructions, discharge instructions, prescriptions, etc., for a typical knee replacement procedure as performed by that surgeon.

In another embodiment, disclosed is a computer system and method that automatically creates medical documents, such as post-operative documents via a plurality of touch screen units. Generated on a GUI of a touch screen unit is a user interactive animated visual representation of at least a portion of a human body representative of the patient including a plurality of selectable anatomical anomalies that may be relevant to the patient. Once one or more portions of the human body portions are selected, one or more medical procedures that may be associated with respect to the selected human body portions are displayed on the GUI. A user then selects, via the GUI, one or more medical procedures performed on the patient with respect to the selected human body portions. A surgical report is then automatically generated for the one or more selected performed medical procedures. One or more Artificial Intelligence (AI) techniques are utilized for determining the one or more medical procedures identified on the GUI and/or for generating the medical report.

### BRIEF DESCRIPTION OF THE DRAWINGS

So that those skilled in the art to which the subject disclosure appertains will readily understand how to make and use the devices and methods of the subject disclosure without undue experimentation, preferred illustrated embodiments thereof will be described in detail herein below with reference to certain figures, wherein:

FIG. 1 illustrates an example communication network utilized with one or more of the illustrated embodiments;

FIG. 2 illustrates an example network device/node utilized with one or more of the illustrated embodiments;

FIG. 3 illustrates a diagram depicting an Artificial Intelligence (AI) device utilized with one or more of the illustrated embodiments;

FIG. 4 illustrates a diagram depicting an AI server utilized with one or more of the illustrated embodiments;

FIG. 5 is a schematic three-dimensional, perspective view of a surgical suite;

FIG. 6 is a function diagram of a system in accordance with an illustrated embodiment;

FIG. 7 is a flow diagram of a method in accordance with an illustrated embodiment;

FIG. 8 is a schematic illustration of a hierarchical tree structure for organizing related surgical procedures into a group in accordance with an illustrated embodiment;

FIG. 9 is a schematic illustration of a data structure in accordance with an illustrated embodiment;

FIG. 10 is an illustration of a form medical document in accordance with an illustrated embodiment;

FIG. 11 is a schematic illustration of a data structure in accordance with an illustrated embodiment;

FIGS. 12A-12D are flow diagrams of a method in accordance with an illustrated embodiment;

FIG. 13 is an illustration of a graphical user interface in accordance with an illustrated embodiment;

FIG. 14 is an illustration of a graphical user interface in accordance with an illustrated embodiment;

FIG. 15 is an illustration of a graphical user interface in accordance with an illustrated embodiment;

FIG. 16 is a flow diagram of a method in accordance with an illustrated embodiment;

FIG. 17 is a flow diagram of a method in accordance with an illustrated embodiment;

FIG. 18 is an illustration of a scheduling dashboard in accordance with an illustrated embodiment;

FIG. 19 is a schematic illustration of a graphical user interface in accordance with an illustrated embodiment;

FIG. 20 is a schematic illustration of a graphical user interface in accordance with an illustrated embodiment;

FIG. 21 illustrates a flow process for a system in accordance with another illustrated embodiment for generating medical records; and

FIGS. 22A-22E illustrate user interactive screen shoots for generating medical records in accordance with the illustrated embodiments.

### DESCRIPTION OF CERTAIN EMBODIMENTS

The illustrated embodiments are now described more fully with reference to the accompanying drawings wherein like reference numerals identify similar structural/functional features. The illustrated embodiments are not limited in any way to what is illustrated as the illustrated embodiments described below are merely exemplary, which can be embodied in various forms, as appreciated by one skilled in the art. Therefore, it is to be understood that any structural and functional details disclosed herein are not to be interpreted as limiting, but merely as a basis for the claims and as a representation for teaching one skilled in the art to variously employ the discussed embodiments. Furthermore, the terms and phrases used herein are not intended to be limiting but rather to provide an understandable description of the illustrated embodiments.

Unless defined otherwise, all technical and scientific terms used herein have the same meaning as commonly understood by one of ordinary skill in the art to which these illustrated embodiments belong. Although any methods and materials similar or equivalent to those described herein can also be used in the practice or testing of the illustrated embodiments, exemplary methods and materials are now described.

It must be noted that as used herein and in the appended claims, the singular forms “a”, “an,” and “the” include plural referents unless the context clearly dictates otherwise. Thus, for example, reference to “a stimulus” includes a plurality of such stimuli and reference to “the signal” includes reference to one or more signals and equivalents thereof known to those skilled in the art, and so forth.

It is to be appreciated the illustrated embodiments discussed below preferably include a software algorithm, program or code residing on computer useable medium having control logic for enabling execution on a machine having a computer processor. The machine typically includes memory storage configured to provide output from execution of the computer algorithm or program.

As used herein, the term “software” is meant to be synonymous with any code or program that can be in a processor of a host computer, regardless of whether the implementation is in hardware, firmware or as a software computer product available on a disc, a memory storage device, or for download from a remote machine. The embodiments described herein include such software to implement the equations, relationships and algorithms described above. One skilled in the art will appreciate further features and advantages of the illustrated embodiments based on the above-described embodiments. Accordingly, the illustrated embodiments are not to be limited by what has been particularly shown and described, except as indicated by the appended claims.

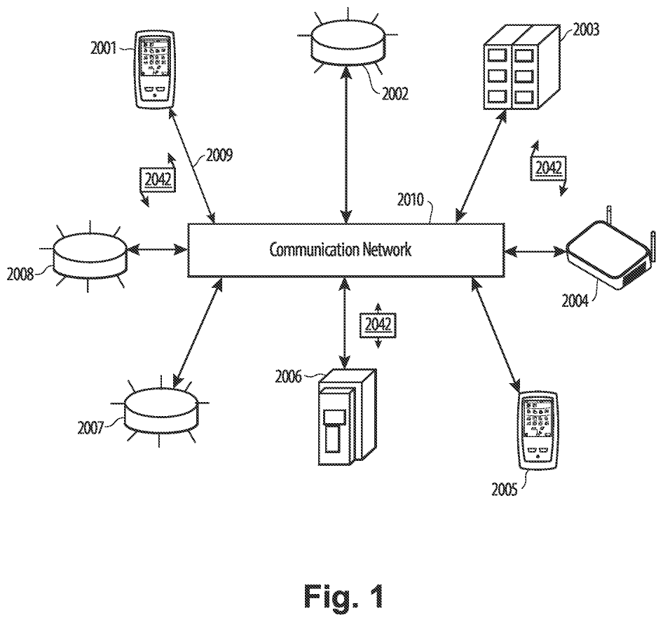

Turning now descriptively to the drawings, in which similar reference characters denote similar elements throughout the several views, FIG. 1 depicts an exemplary communications network 2010 in which below illustrated embodiments may be implemented. It is to be understood a communication network 2010 is a geographically distributed collection of nodes interconnected by communication links and segments for transporting data between end nodes, such as personal computers, work stations, smart phone devices, tablets, televisions, sensors and or other devices such as automobiles, etc. Many types of networks are available, with the types ranging from local area networks (LANs) to wide area networks (WANs). LANs typically connect the nodes over dedicated private communications links located in the same general physical location, such as a building or campus. WANs, on the other hand, typically connect geographically dispersed nodes over long-distance communications links, such as common carrier telephone lines, optical lightpaths, synchronous optical networks (SONET), synchronous digital hierarchy (SDH) links, or Powerline Communications (PLC), and others.

FIG. 1 is a schematic block diagram of an example communication network 2010 illustratively comprising nodes/devices 2001-2008 (e.g., smart computing devices 2001, sensors 2002, client computing devices 2003, smart phone devices 2005, web servers 2006, routers 2007, switches 2008, databases, and the like) interconnected by various methods of communication. For instance, the links 2009 may be wired links or may comprise a wireless communication medium, where certain nodes are in communication with other nodes, e.g., based on distance, signal strength, current operational status, location, etc. Moreover, each of the devices can communicate data packets (or frames) 2042 with other devices using predefined network communication protocols as will be appreciated by those skilled in the art, such as various wired protocols and wireless protocols etc., where appropriate. In this context, a protocol consists of a set of rules defining how the nodes interact with each other. Those skilled in the art will understand that any number of nodes, devices, links, etc. may be used in the computer network, and that the view shown herein is for simplicity. Also, while the embodiments are shown herein with reference to a general network cloud, the description herein is not so limited, and may be applied to networks that are hardwired.

As will be appreciated by one skilled in the art, aspects of the illustrated embodiments may be embodied as a system, method or computer program product. Accordingly, aspects of the illustrated embodiments may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the illustrated embodiments may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device. A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the illustrated embodiments may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the illustrated embodiments are described below with reference to flowchart illustrations and/or block diagrams of methods, apparatus (systems) and computer program products according to the illustrated embodiments. It will be understood that each block of the flowchart illustrations and/or block diagrams, and combinations of blocks in the flowchart illustrations and/or block diagrams, can be implemented by computer program instructions. These computer program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

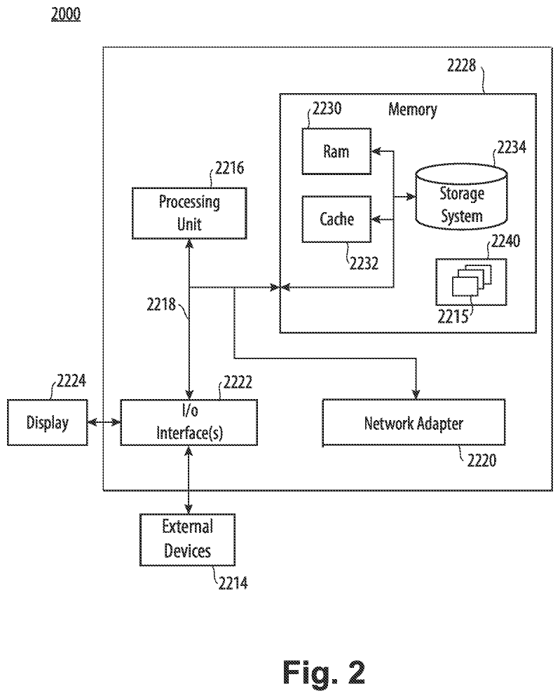

FIG. 2 is a schematic block diagram of an example network computing device 2000 (e.g., client computing device 2003, server 2006, etc.) that may be used (or components thereof) with one or more embodiments described herein, e.g., as one of the nodes shown in the network 2010, including control system 100 described further below. As explained above, in different embodiments these various devices are configured to communicate with each other in any suitable way, such as, for example, via communication network 2010.

Device 2000 is intended to represent any type of computer system capable of carrying out the teachings of various illustrated embodiments. Device 2000 is only one example of a suitable system and is not intended to suggest any limitation as to the scope of use or functionality of the illustrated embodiments described herein. Regardless, computing device 2000 is capable of being implemented and/or performing any of the functionality set forth herein.

Computing device 2000 is operational with numerous other general purpose or special purpose computing system environments or configurations. Examples of well-known computing systems, environments, and/or configurations that may be suitable for use with computing device 2000 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, and distributed data processing environments that include any of the above systems or devices, and the like. Computing device 2000 may be described in the general context of computer system-executable instructions, such as program modules, being executed by a computer system. Generally, program modules may include routines, programs, objects, components, logic, data structures, and so on that perform particular tasks or implement particular abstract data types. Computing device 2000 may be practiced in distributed data processing environments where tasks are performed by remote processing devices that are linked through a communications network. In a distributed data processing environment, program modules may be located in both local and remote computer system storage media including memory storage devices. In accordance with the illustrated embodiments, computing device 2000 is configured and operative to generate one or more medical documents utilizing hierarchically organized database views presented on a touch screen interface for automatically generating medical reports indicative of one or more medical (e.g., surgical) procedures performed on a patient.

The components of device 2000 may include, but are not limited to, one or more processors or processing units 2216, a system memory 2228, and a bus 2218 that couples various system components including system memory 2228 to processor 2216. Bus 2218 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus. Computing device 2000 typically includes a variety of computer system readable media. Such media may be any available media that is accessible by device 2000, and it includes both volatile and non-volatile media, removable and non-removable media.

System memory 2228 can include computer system readable media in the form of volatile memory, such as random-access memory (RAM) 2230 and/or cache memory 2232. Computing device 2000 may further include other removable/non-removable, volatile/non-volatile computer system storage media. By way of example only, storage system 2234 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 2218 by one or more data media interfaces. As will be further depicted and described below, memory 2228 may include at least one program product having a set (e.g., at least one) of program modules that are configured to carry out the functions of embodiments of illustrated embodiments.

Program/utility 2240, having a set (at least one) of program modules 2215, such as underwriting module, may be stored in memory 2228 by way of example, and not limitation, as well as an operating system, one or more application programs, other program modules, and program data. Each of the operating system, one or more application programs, other program modules, and program data or some combination thereof, may include an implementation of a networking environment. Program modules 2215 generally carry out the functions and/or methodologies of the illustrated embodiments as described herein, including generating one or more medical documents utilizing hierarchically organized database views presented on a touch screen interface for automatically generating medical reports indicative of one or more medical (e.g., surgical) procedures performed on a patient.

Device 2000 may also communicate with one or more external devices 2214 such as a keyboard, a pointing device, a display 2224, etc.; one or more devices that enable a user to interact with computing device 2000; and/or any devices (e.g., network card, modem, etc.) that enable computing device 2000 to communicate with one or more other computing devices. Such communication can occur via Input/Output (I/O) interfaces 2222. Still yet, device 2000 can communicate with one or more networks such as a local area network (LAN), a general wide area network (WAN), and/or a public network (e.g., the Internet) via network adapter 2220. As depicted, network adapter 2220 communicates with the other components of computing device 2000 via bus 2218. It should be understood that although not shown, other hardware and/or software components could be used in conjunction with device 2000. Examples, include, but are not limited to: microcode, device drivers, redundant processing units, external disk drive arrays, RAID systems, tape drives, and data archival storage systems, etc.

FIGS. 1 and 2 are intended to provide a brief, general description of an illustrative and/or suitable exemplary environment in which the below described illustrated embodiments may be implemented. FIGS. 1 and 2 are exemplary of a suitable environment and are not intended to suggest any limitation as to the structure, scope of use, or functionality of an illustrated embodiment. A particular environment should not be interpreted as having any dependency or requirement relating to any one or combination of components illustrated in an exemplary operating environment. For example, in certain instances, one or more elements of an environment may be deemed not necessary and omitted. In other instances, one or more other elements may be deemed necessary and added.

It is to be understood the embodiments described herein are preferably provided with self-learning/Artificial Intelligence (AI) for generating one or more medical documents utilizing hierarchically organized database views presented on a touch screen interface for automatically generating medical reports indicative of one or more medical (e.g., surgical) procedures performed on a patient, as well as one or more determinations relating to a medical procedure as described herein (including, but not limited to: i) anatomical anomalies relating to a particular patient; ii) medical procedures, and subsets of medical procedures that may be performed relative to a patient; iii) surgical instrumentalities that may be used for a particular medical procedure relating to a particular patient; iv) pharmaceuticals that may be used for a particular medical procedure relating to a particular patient that may be performed; pharmaceuticals that may be prescribed post a particular medical procedure relating to a particular patient; that may be performed; and vi) one or more item contents included in a generated medical report (e.g., appropriate billing codes).

Preferably integrated into a computer system 2000, coupled to a plurality of external databases/data sources, is an AI system (e.g., an Expert System) that implements machine learning and artificial intelligence algorithms to conduct one or more of the above mentioned tasks, including generating one or more medical documents utilizing hierarchically organized database views presented on a touch screen interface for automatically generating medical reports indicative of one or more medical (e.g., surgical) procedures performed on a patient. For instance, the AI system may include two subsystems: a first sub-system that learns from historical data; and a second subsystem to identify and recommend one or more parameters or approaches based on the learning. It should be appreciated that although the AI system may be described as two distinct subsystems, the AI system can also be implemented as a single system incorporating the functions and features described with respect to both subsystems.

In accordance with the illustrated embodiments described herein, artificial intelligence refers to the field of studying artificial intelligence or methodology for making artificial intelligence, and machine learning refers to the field of defining various issues dealt with in the field of artificial intelligence and studying methodology for solving the various issues. Machine learning is defined as an algorithm that enhances the performance of a certain task through a steady experience with the certain task.

Also in accordance with the illustrated embodiments, an artificial neural network (ANN) is a model used in machine learning and may mean a whole model of problem-solving ability which is composed of artificial neurons (nodes) that form a network by synaptic connections. The artificial neural network can be defined by a connection pattern between neurons in different layers, a learning process for updating model parameters, and an activation function for generating an output value. The artificial neural network may include an input layer, an output layer, and optionally one or more hidden layers. Each layer includes one or more neurons, and the artificial neural network may include a synapse that links neurons to neurons. In the artificial neural network, each neuron may output the function value of the activation function for input signals, weights, and deflections input through the synapse.

Model parameters refer to parameters determined through learning and include a weight value of synaptic connection and deflection of neurons. A hyperparameter means a parameter to be set in the machine learning algorithm before learning, and includes a learning rate, a repetition number, a mini batch size, and an initialization function. The purpose of the learning of the artificial neural network may be to determine the model parameters that minimize a loss function. The loss function may be used as an index to determine optimal model parameters in the learning process of the artificial neural network. Machine learning may be classified into supervised learning, unsupervised learning, and reinforcement learning according to a learning method. The supervised learning may refer to a method of learning an artificial neural network in a state in which a label for learning data is given, and the label may mean the correct answer (or result value) that the artificial neural network must infer when the learning data is input to the artificial neural network. The unsupervised learning may refer to a method of learning an artificial neural network in a state in which a label for learning data is not given. The reinforcement learning may refer to a learning method in which an agent defined in a certain environment learns to select a behavior or a behavior sequence that maximizes cumulative compensation in each state.

Machine learning, which is implemented as a deep neural network (DNN) including a plurality of hidden layers among artificial neural networks, is also referred to as deep learning, and the deep learning is part of machine learning.

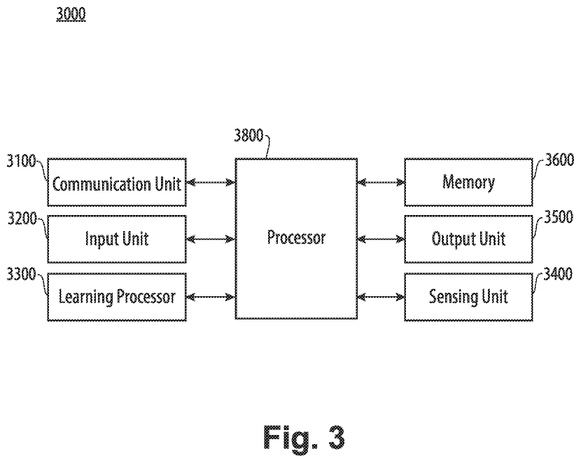

FIG. 3 illustrates an AI device 3000 according to an embodiment of the illustrated embodiments. In accordance with the illustrated embodiments, the AI device 3000 may be implemented by a computer system 2000 for generating one or more medical documents utilizing hierarchically organized database views presented on a touch screen interface for automatically generating medical reports indicative of one or more medical (e.g., surgical) procedures performed on a patient.

Referring to now FIG. 3, in conjunction with FIGS. 1 and 2, the AI device 3000 is operatively coupled to, or integrated with computing device 2000, in accordance with the illustrated embodiments described herein. AI device 300 preferably includes a communication unit 3310, an input unit 3320, a learning processor 3330, a sensing unit 3340, an output unit 3350, a memory 3370, and a processor 3380. The communication unit 3310 may transmit and receive data to and from external devices such as other AI devices 3300a to 3300e and the AI server 4000 by using wire/wireless communication technology. For example, the communication unit 3310 may transmit and receive sensor information, a user input, a learning model, and a control signal to and from external devices.

The communication technology used by the communication unit 3310 preferably includes GSM (Global System for Mobile communication), CDMA (Code Division Multi Access), LTE (Long Term Evolution), 5G, WLAN (Wireless LAN), Wi-Fi (Wireless-Fidelity), Bluetooth™, RFID (Radio Frequency Identification), Infrared Data Association (IrDA), ZigBee, NFC (Near Field Communication), and the like.

The input unit 3200 may acquire various kinds of data, including, but not limited to information relating to a patient (including the patient's medical history), information regarding medical procedures (including surgeries) and numerous other types of information having relevancy to one or both of a patient and medical procedures performed on a patient. The input unit 3200 may acquire learning data for model learning and an input data to be used when an output is acquired by using learning model. The input unit 3200 may acquire raw input data. In this case, the processor 3800 or the learning processor 3300 may extract an input feature by preprocessing the input data. The learning processor 3300 may learn a model composed of an artificial neural network by using learning data. The learned artificial neural network may be referred to as a learning model. The learning model may be used to an infer result value for new input data rather than learning data, and the inferred value may be used as a basis for determination to perform a certain operation. In accordance with certain illustrated embodiments, the aforesaid learning processor, for generating a medical report, as described herein, includes usage of a Large Language Model (LLM).

At this time, the learning processor 3300 may perform AI processing together with the learning processor 3300 of the AI server 4000, and the learning processor 3300 may include a memory integrated or implemented in the AI device 3000. Alternatively, the learning processor 330 may be implemented by using the memory 3700, an external 20) memory directly connected to the AI device 300, or a memory held in an external device. The sensing unit 340 may acquire at least one of internal information about the AI device 300, ambient environment information about the AI device 300, and user information by using various sensors.

The output unit 3500 preferably includes a display unit for outputting/displaying relevant information to a user in accordance with the illustrated embodiments described herein. The memory 3700 preferably stores data that supports various functions of the AI device 3000. For example, the memory 3700 may store input data acquired by the input unit 320, learning data, a learning model, a learning history, and the like.

In accordance with certain illustrated embodiments, the AI device 3000 and/or memory 3700, that is operatively coupled to the below described computer server 132 is remotely located from the computer server 132, such as a “cloud based” system. Thus and as described further below, the computer server 132 is operatively couped to a plurality of unaffiliated hospital/medical facilities (e.g., 100), providing the same, or substantially the same, functionality to each location (e.g., 100) having one or more input units (e.g., 116) for causing a medical report to be generated as described in accordance with the illustrated embodiments. For instance, medical procedural data utilized by an end user (e.g., a surgeon) may optionally be collected into a procedure database (e.g., memory 3700 and/or 2228) which is cloud connected to a plurality of users (e.g., medical facilities 100) whereby AI is utilized to suggest medical/surgical procedural steps used by a plurality of surgeons, in various worldwide locations. Additionally, data input providing various relevant medical information (e.g., up to date medical publication information) may be populated into the database (e.g., memory 3700 and/or 2228).

The processor 3800 preferably determines at least one executable operation of the AI device 3000 based on information determined or generated by using a data analysis algorithm or a machine learning algorithm. The processor 3800 may control the components of the AI device 3000 to execute the determined operation. To this end, the processor 3800 may request, search, receive, or utilize data of the learning processor 3300 or the memory 3700. The processor 3800 may control the components of the AI device 3000 to execute the predicted operation or the operation determined to be desirable among the at least one executable operation. When the connection of an external device is required to perform a determined operation, the processor 3800 may generate a control signal for controlling the external device and may transmit the generated control signal to the external device. The processor 3800 may acquire intention information for the user input and may determine the user's requirements based on the acquired intention information.

The processor 3800 may acquire the intention information corresponding to the user input by using at least one of a speech to text (STT) engine for converting speech input into a text string or a natural language processing (NLP) engine for acquiring intention information of a natural language.

At least one of the STT engine or the NLP engine may be configured as an artificial neural network, at least part of which is learned according to the machine learning algorithm. At least one of the STT engine or the NLP engine may be learned by the learning processor 3300, may be learned by the learning processor 3400 of the AI server 4000, or may be learned by their distributed processing. The processor 3800 may collect history information including the operation contents of the AI device 3000 or the user's feedback on the operation and may store the collected history information in the memory 3700 or the learning processor 3300 or transmit the collected history information to the external device such as the AI server 4000. The collected history information may be used to update the learning model.

The processor 3800 may control at least part of the components of AI device 3000 so as to drive an application program stored in memory 3700. Furthermore, the processor 3800 may operate two or more of the components included in the AI device 3000 in combination so as to drive the application program.

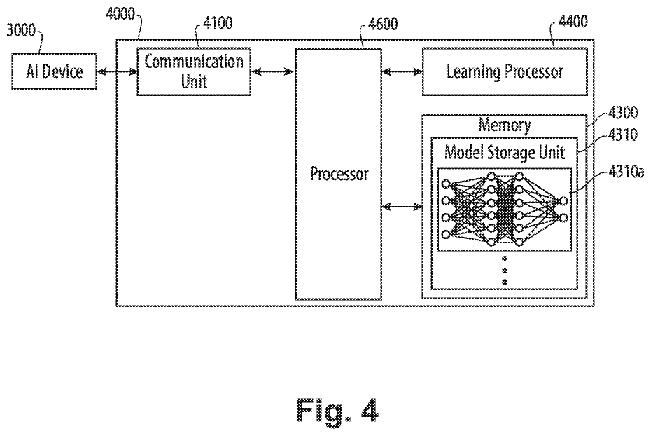

FIG. 4 illustrates an AI server 4000 according to the illustrated embodiments. It is to be appreciated that the AI server 4000 may refer to a device that learns an artificial neural network by using a machine learning algorithm or uses a learned artificial neural network. The AI server 4000 may include a plurality of servers to perform distributed processing, or may be defined as a 5G network. At this time, the AI server 4000 may be included as a partial configuration of the AI device 3000, and may perform at least part of the AI processing together. The AI server 4000 may include a communication unit 4100, a memory 4300, a learning processor 4400, a processor 4600, and the like. The communication unit 4100 can transmit and receive data to and from an external device such as the AI device 3000. The memory 4300 may include a model storage unit 4310. The model storage unit 4310 may store a learning or learned model (or an artificial neural network 4310a) through the learning processor 4400.

The learning processor 4400 may learn the artificial neural network 4310a by using the learning data. The learning model may be used in a state of being mounted on the AI server 4000 of the artificial neural network, or may be used in a state of being mounted on an external device such as the AI device 3000. The learning model may be implemented in hardware, software, or a combination of hardware and software. If all or part of the learning models are implemented in software, one or more instructions that constitute the learning model may be stored in memory 4300. The processor 4600 may infer the result value for new input data by using the learning model and may generate a response or a control command based on the inferred result value.

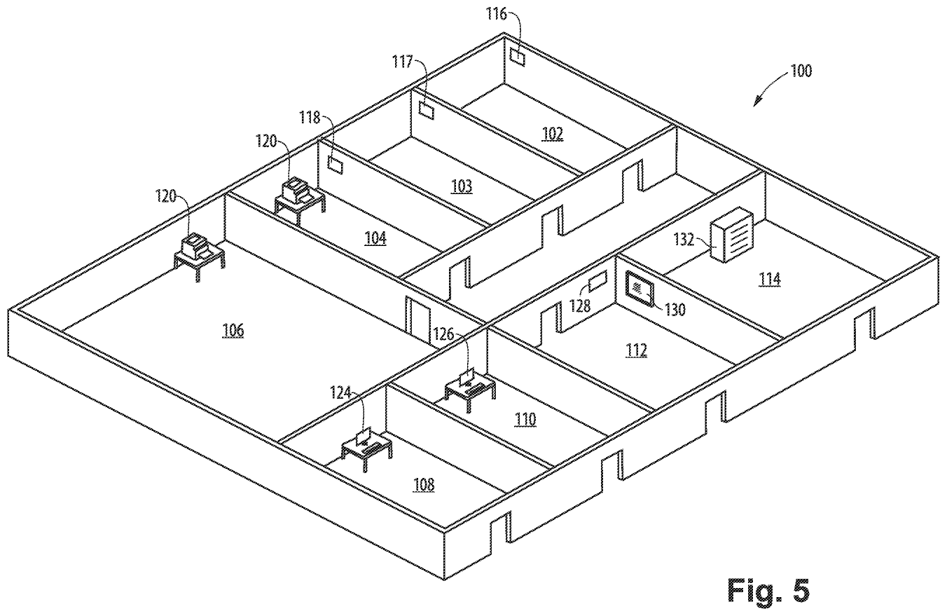

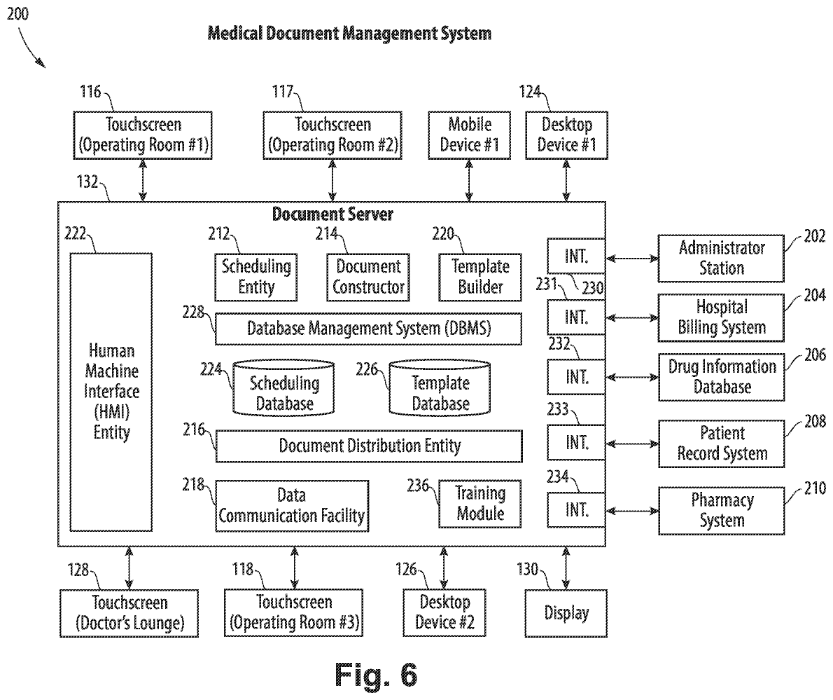

With the exemplary communication network 2010 (FIG. 1), computing device 2000 (FIG. 2), AI device 3000 (FIG. 3) and AI server 4000 (FIG. 4) being generally shown and discussed above, description of certain illustrated embodiments will now be provided with reference to FIGS. 5 and 6, which incorporate one or more components of above described systems of FIGS. 1-4. It is to be understood and appreciated that FIGS. 1-6 are intended to provide a brief, general description of an illustrative and/or suitable exemplary environment in which the below described illustrated embodiments may be implemented. FIGS. 1-6 are exemplary of a suitable environment and are not intended to suggest any limitation as to the structure, scope of use, or functionality of an illustrated embodiment. A particular environment should not be interpreted as having any dependency or requirement relating to any one or combination of components illustrated in an exemplary operating environment. For example, in certain instances, one or more elements of an environment may be deemed not necessary and omitted. In other instances, one or more other elements may be deemed necessary and added.

With reference now to FIG. 5, shown is a schematic, three-dimensional (3D) perspective view of a surgical suite 100, which may be located in a hospital or other medical facility. The surgical suite 100 may include a plurality of operating rooms, such as operating rooms 102-104. The suite 100 also may include a recovery room 106, one or more doctor's offices, such as offices 108 and 110, a doctors' lounge 112, and an information technology (IT) and/or data center 114. Within each operating room 102-104 may be a input units 116-118. For example, the input units may be touchscreen units 116-118 (e.g., that may be wall mounted within the respective operating rooms 102-104). In accordance with the illustrated embodiments, the touchscreen units 116-118 may consist of a portable smart device (e.g., 2001 of FIG. 1, such as, but not limited to: a smart phone device, smart tablet device and other suitable portable computing devices (examples being an iOS or Android smart phone and/or tablet device) having a touch screen user interface and software (e.g., an app) installed and executable to communicate with below described computer server 132 for providing the electronic data functionality described herein for generating medical reports.

In certain illustrated embodiments, one or more of the input touchscreen units 116-118 consists of stylus and associated touchscreen units, which are to be understood to be preferably handheld electronic devices having a writing (e.g., soft) tip configured and operative to operate a cooperating touch screen unit. For instance, they may include a passive stylus pen, also known as a capacitive stylus, that register input on a touchscreen by either distorting the touchscreen's electrostatic field or blocking the transmission of light. Additionally, an active stylus pen may be utilized which utilizes internal communication technology to perform touch commands. Active styluses typically contain computer chips, or other hardware, inside of them, which they use to communicate with touchscreens. Further, a Bluetooth stylus device may be utilized, as well as an Apple Pencil stylus device.

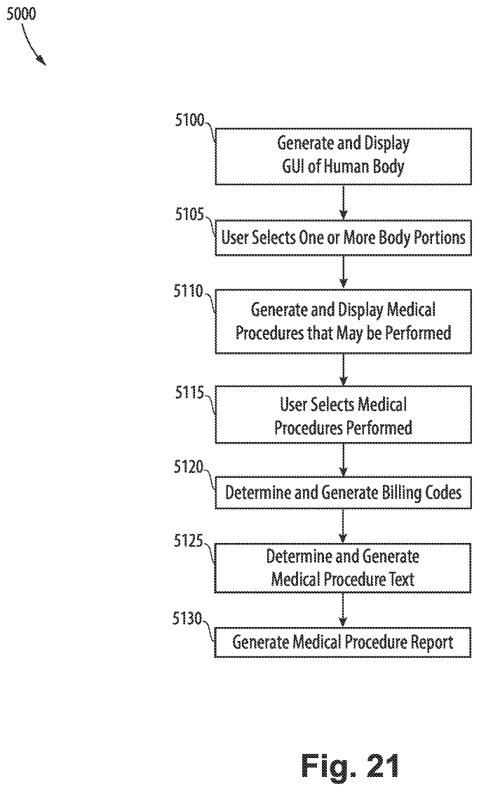

In other illustrated embodiments, the input units 116-118 utilize voice recognition techniques (e.g., dictation techniques) for enabling a user (e.g., a surgeon) to provide input, or otherwise interact with a GUI, as described herein for generating a medical report as described below with reference 5000 of FIG. 21.

In yet additional illustrated embodiments, one or more of the input units 116-118 consists of a virtual reality (VR) headset, also known as VR goggles, which is commonly known as a head-mounted device that provides immersive 3D virtual experiences, that typically includes a pair of lenses that users look through, a screen (or screens) inside the device that the user interacts with, a mechanism to secure it to the user's head, and VR input units (e.g., handheld devices, the user's hands/fingers, etc.) configured and operative to enable the user to interact with GUI's provided in the VR screen In accordance with the illustrated embodiments, the computer system 132 causes the below described GUI's to be generated in a VR screen to which the user interacts with as described further below with reference to process 5000 of FIG. 21. Additionally, the one or more of the operating rooms 102-104 may include a printer, such as printer 120 located in operating room 104. Another printer, such as printer 122, may be located in the recovery room 106. Within each of the doctor's offices 108 and 110 may be a computer, such as desktop computers 124 and 126. Another touchscreen unit, such as touchscreen unit 128, as well as a display 130, which may be operated as a dashboard, may be located in the doctors' lounge 112.

One or more servers, such as a document computer server 132 (including one or more components of the computer systems shown as described in FIGS. 1-4), may be locally located relative to the hospital or other medical facility (e.g., in the IT and/or data center 114). However, it is to be understood and appreciated the computer server 132 is preferably geographically remotely located from the hospital or other medical facility (e.g., the surgical suite 100) and is communicatively coupled thereto via a communications network 2010. For instance, the document server 132 may be a cloud-based system that is couped to a plurality of other hospital/medical facilities, providing the same functionality as described with reference to surgical suite 100.

With reference now to FIG. 6 (and with continuing reference to FIGS. 1-5) shown is a schematic illustration of a medical document management system 200 for, among other things, automatically creating post-operative documents. The system 200 includes the document server 132. The document server 132 is configured to communicate with a plurality of devices. The devices may include: the touchscreen units 116-118 and 128 located in the operating rooms 102-104 and in the doctors' lounge 112; the desktop computers 124 and 126 located in the doctors' offices 108 and 110; and the dashboard 130 located in the doctors' lounge 112. The document server 132 also may communicate with an administrator station 202, a hospital billing system 204, a drug information database 206, a patient record system 208, and a pharmacy system 210.

The document server 132 itself may include a plurality of components. Specifically, the server 132 may include a scheduling entity 212, a document constructor 214, a document distribution entity 216, a data communication facility 218, a template builder 220, and a human-machine interface (HMI) entity 222. In addition, the document server 232 may include a scheduling database 224, a template database 226, and one or more database management systems 228 that interface to the scheduling and template databases 224, 226. The document server 132 also may include one or more interfaces, such as interfaces 230-234 that interface to the administrator station 202, hospital billing system 204, drug information database 206, patient record system 208, and pharmacy system 210. The document server 132 also may include a training or learning module 236, which preferably utilizes one or more AI/ML techniques via system 3000 (FIG. 3) and the AI server 4000 (FIG. 4).

Suitable servers 132 for use with the present invention include the ProLiant and Integrity series of servers from Hewlett Packard Co. of Palo Alto, CA, and the PowerEdge series of servers from Dell Inc. of Round Rock, TX, among others.

Nonetheless, those skilled in the art will understand that the document server 132 is meant for illustrative purposes only, and that the present invention may be used with other computer systems, processing systems or computational devices.

And as mentioned above, suitable touch screen units include the iPad series of tablets from Apple, Inc. of Cupertino, CA, the Galaxy Tab series of tablets from Samsung Electronics Co., Ltd. of Seoul, South Korea, and the Kindle Fire series of tablets from Amazon.com, Inc. of Seattle, WA. Other touch screen units may include smart phones, such as the iPhone series of smart phones from Apple, Inc.

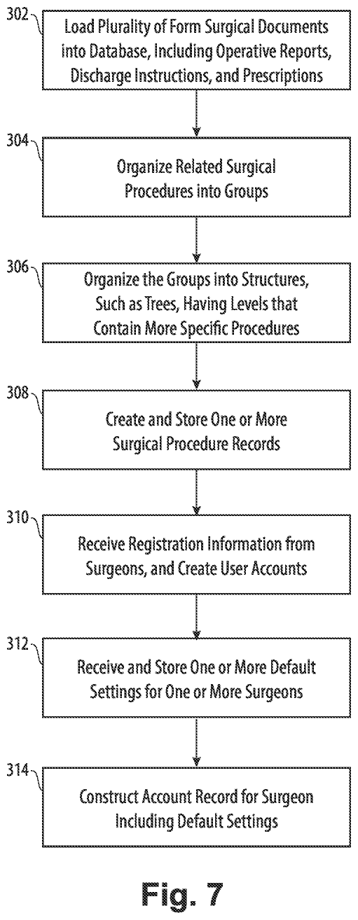

FIG. 7 is a flow diagram of a method in accordance with an embodiment of the present invention. A system administrator may load predefined form documents for various surgical procedures into the template database 226, as indicated at block 302. For example, the system administrator may create form operative reports, form recovery room instructions, and form discharge instructions for a plurality of surgical procedures, such as carpal tunnel release, hernia repair, hip replacement, knee replacement, appendectomy, etc. These predefined form documents may be uploaded to the document server 132, and stored in the template database 226. One or more of the forms may include placeholders that may be replaced with particular information when a document, based on the form, is created. For example, one or more of the form operative reports may include placeholders for such items as the patient name, the type of anesthesia used during the procedure, the patient's estimated blood loss, the amount of intravenous fluids provided to the patient during the procedure, etc.

In an embodiment, related surgical procedures for which information, such as one or more documents, is stored at the template database 226 are organized into a group or set, as indicated at block 304. In addition, each group or set of related surgical procedures may be arranged in a hierarchical structure, such as a tree, having a plurality of levels, including a root level, as indicated at block 306.

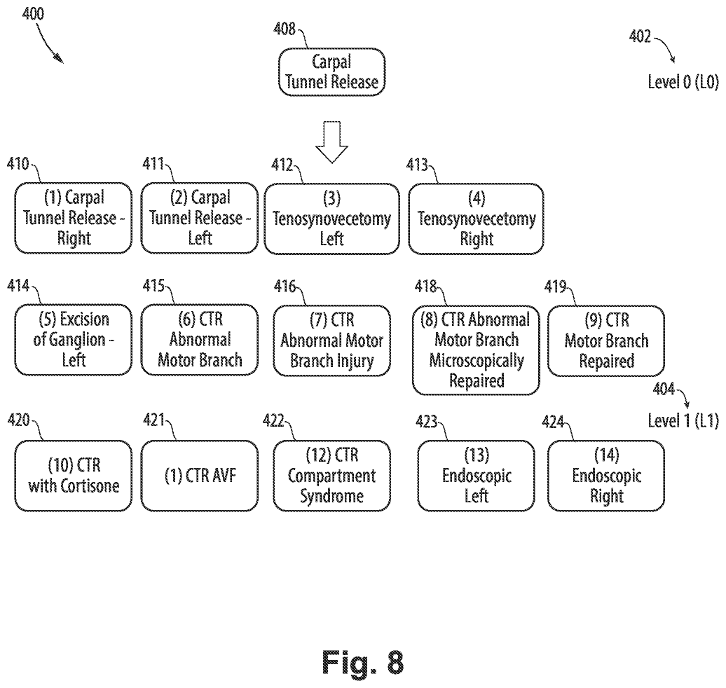

FIG. 8 is a schematic illustration of a hierarchical tree structure 400 for a group or set of related surgical procedures. The tree structure 400 has a plurality of levels including a root level (Level 0) 402, and a first level (Level 1) 404. The root level 402 may have a single, root node 408 that specifies a highest level, e.g., a generalized description of the group of related surgical procedures, e.g., carpal tunnel release (CTR). The tree 400 also may include one or more Level 1 (L1) nodes 410-424 that may be logically connected to the root node 408 as child nodes. The L1 nodes 410-424 may be located within the first level (L1) 404 of the tree 400. Each L1 node 410-424 may specify a surgical procedure at a next level of specificity from the generalized procedure specified at the root node 408. As an example, the L1 nodes 410-424 may specify Carpal Tunnel Release—Right, Carpal Tunnel Release—Left, Tenosynovecetomy—Right, Tenosynovecetomy—Left, Excision of Ganglion—Left, CTR Abnormal Motor Branch, CTR Abnormal Motor Branch Injury, CTR Abnormal Motor Branch Microscopically Repaired, CTR Motor Branch Repaired, CTR with Cortisone, CTR AVF, CTR Compartment Syndrome, Endoscopic-Left, and Endoscopic-Right, respectively.

In an embodiment, one or more of the L1 nodes 410-424 may have one or more Level 2 (L2) nodes. Each L2 node may be located within a second level (L2) of the tree 400, and may be connected to a particular L1 node as a child node of that L1 node. In addition, one or more nodes located within a third level (L3) may be connected to one or more of the L2 nodes. Each L3 node may specify a surgical procedure at a next level of specificity from the surgical procedure of its parent node, i.e., its L2 node. It should be understood that some of the nodes at a given level may not have any child nodes, while other nodes at the given level may have child nodes. The tree 400 organizes a group of related surgical procedures in increasing level of specificity by virtue of the levels and the connections between parent and child nodes.

The level one (L1) nodes as well as the child nodes for a given parent node May each be assigned a rank order. In an embodiment, the L1 and the child nodes are ranked from most common procedure to least common procedure. A most common procedure is one that occurs most frequently within a group, whereas a least common procedure occurs least often. The ranking thus provides an indication of how frequently the respective procedure occurs. For the L1 nodes, Carpal Tunnel Release-right (rank #1) is the most frequently performed procedure in this group, while endoscopic-right (rank #14) is the least frequently performed procedure in this group.

Other trees may be similarly established for other groups or sets of related surgical procedures. For example, for a retrocecal appendix, the root level (L0) may be retrocecal appendix, and the level 1 (L1) nodes may be Appendectomy Open, Laparoscopy Appendectomy, Appendectomy Purse String, Appendectomy Z Stitch, Appendectomy Stump Not Buried, Appendectomy Stapled, Appendectomy for Perforated Appendix, Appendectomy with CA, Appendectomy Excision of Mass, Appendectomy CA Colon, Ectopic Pregnancy, Normal Appendix, Meckel's Diverticulitis, and Lap to Open.

In addition, other data structures besides hierarchical trees may be employed. For example, one or more spreadsheets may be used to establish the relationships among parent, child and root-level procedures.

The groups or sets of related procedures and their relationship as parent and child procedures may be determined based on the knowledge of highly experienced surgeons in the respective field or specialty. For example, one or more experienced hand surgeons may be interviewed to gather the information concerning the possible procedures and complications that can arise during, for example, a carpal tunnel release. Surgeons having sufficient experience in other specialties, such as hernia repair, appendectomy, heart surgery, etc., may be interviewed and groups of related procedures, parent/child relationships, findings, specimens, skin closures, and complications may be obtained, and the information may be stored in the template database 626.

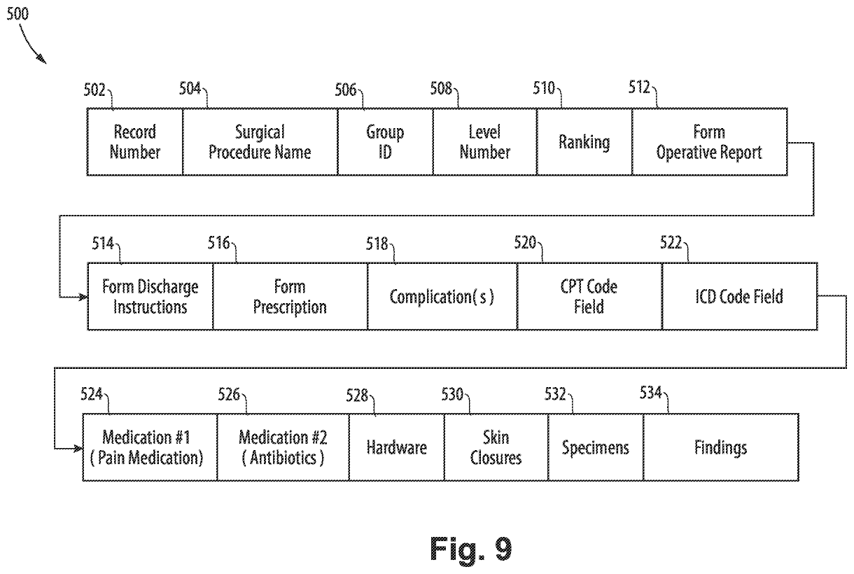

In an embodiment, the database management system 228 may create one or more records in the template database 226 for each surgical procedure, as indicated at block 308. FIG. 9 is a schematic illustration of a surgical procedure record 500 created by the database management system 228, and stored for example in the template database 226. The record 500 may include a plurality of fields or cells configured to store information. Specifically, the record 500 may include a record number field 502 that may contain a value identifying the record 500 within the database 226, a surgical procedure name field 504 that contains information identifying the particular surgical procedure, such as the commonly accepted name of the procedure, e.g., carpal tunnel release, left side. The record 500 also may include a group identification (ID) field 506 that contains information identifying the particular group or set, e.g., carpal tunnel release, to which the particular surgical procedure belongs. In addition, the record 500 may include a level number field 508 that specifies the level within the tree hierarchy of the group or set to which the particular surgical procedure belongs. In addition, the record 500 may include a ranking or sequence number field 510 that contains information identifying the procedure's ranking or sequence number, e.g., 2, among other procedures in its tree hierarchy level.

The record 500 also may include one or more document fields. For example, the record 500 may include an operative report form field 512 that contains a predefined form operative report for this respective procedure. Alternatively, field 512 may include a link or pointer to the operative report, which may be stored as a file in the template database 226 or in another location. Other document fields may include a form discharge instructions field 514 that contains predefined form discharge instructions (or a link or pointer to such instructions) for the respective procedure, a predefined form prescription field 516 that contains a form prescription (or a link or pointer to a prescription) for post-surgery medications for the respective procedure, such as pain medications, antibiotics, etc. Surgical record 500 also may include a complications field 518 that may contain a link or pointer to a predefined list of complications that most often occur when the respective procedure, e.g., carpal tunnel release-right, is performed as well as predefined text describing the complication. The list may be stored in one or more records stored in the template database 226, and managed by the database management system 228. The record 500 also may include one or more fields for storing one or more codes associated with the respective procedure. For example, the record 500 may include a Current Procedural Terminology (CPT) code field 520, and/or an International Statistical Classification of Diseases and Related Health Problems (ICD) code field 522. Fields 520 and 522 may contain values for the respective CPT and ICD codes assigned to the procedure of the surgical record 500.

The record 500 may include one or more fields that store information concerning medications typically prescribed in connection with the respective procedure. For example, the record 500 may include a first medication field 524 that stores information concerning the pain medications that are frequently prescribed to patients following the respective procedure. The information may include the names of the pain medications and the commonly prescribed dosages. Alternatively, the information may be one or more links or pointers to other records or storage locations where the information is held. The record also may include a second medication field 526 that stores information concerning the antibiotics that are frequently prescribed to patients following the respective procedure. The record also may include a hardware field 528 that stores a list of the hardware that may be used in the procedure, such as the number and types of surgical screws, the number and types of plates, etc.

The surgical procedure record 500 may include additional fields, such as a skin closure field 530 that may include a predefined list of skin closures used in the respective procedure (or a link to the same), a specimens field 532 that may include a predefined list of specimens that may be obtained during the respective procedure (or a link to the same), and a findings field 534 that may include a predefined list of findings that may be made during the respective procedure (or a link to the same).

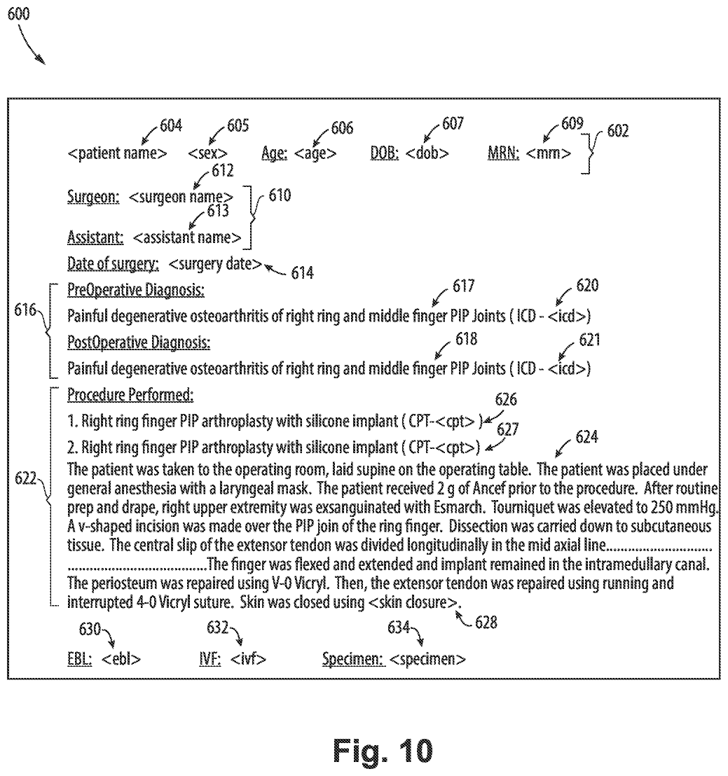

FIG. 10 is a schematic illustration of a form operative report 600 as stored in the template database 226, e.g., at the form operative report field 512 of the respective surgical procedures record 500. The form operative report 600 includes a plurality of placeholders for receiving surgeon specified and patient-specific data. The placeholders are illustrated within report 600 inside brackets (< >). Specifically, the form operative report 600 may include a patient area 602 having a plurality of entries. For example, the patient area 602 may include a patient name placeholder 604, a patient sex placeholder 605, a patient age placeholder 606, a patient date of birth (DOB) placeholder 607, and a patient medical record number (MRN) 608. When creating an actual operative report from the form operative report 600, patient specific data may be obtained and entered into placeholders 604-608. The form operative report 600 also may include a surgeon area 610. The surgeon area may include a surgeon name placeholder 612, and an assistant name placeholder 613. Information to be entered in the surgeon and assistant names placeholders 612, 613 may be obtained from the template database 226. The form operative report 600 may include a date of surgery placeholder 614.

The form operative report 600 also includes predefined information for the respective procedure. In particular, the form operative report 600 may include a diagnosis area 616 that includes a predefined pre-operative diagnosis 617, and a predefined post-operative diagnosis 618. The pre and post operative diagnoses 617, 618 may each include an International Statistical Classification of Diseases and Related Health Problems (ICD) code placeholder 620 and 621, respectively.

The form operative report 600 may further include a procedure area 622 that includes a predefined description 624, which represents the contents or text describing the respective procedure. The predefined description 624 may include one or more placeholders for information concerning an actual procedure. For example, the predefined description 624 may include one or more Current Procedural Terminology (CPT) placeholders, such as first and second CPT placeholders 626 and 627. The predefined description 624 also may include a skin closure placeholder 628. When an actual report is created from the form operative report 600, the actual skin closure used is entered at the skin closure placeholder 628.

The form operative report 600 may include an estimated blood loss (ebl) amount placeholder 630, an intravenous fluid (ivf) amount placeholder 632, and a specimen name placeholder 634.

It should be understood that the form operative report 600 is meant for illustrative purposes only, and that the present invention may be used with form operative reports having alternative formats. For example, other placeholders may be included, such as a findings placeholder, and other predefined information may be included.

In an embodiment, each surgeon utilizing the system 200 may create a respective account by selecting a username and a password, which may be saved by the system 200, as indicated at block 310. In an embodiment, the system 200 may request the surgeon's name, and may assign a username, which may be in the form of <last name, first name>. Thereafter, to log into the system 200, the surgeon may select or enter his or her username and password, and this information may be verified by the system 200. For example, the document server 132 may include a login utility (not shown) that manages usernames and passwords, and controls access to files based on user. Suitable login utilities include Omni Secure Membership Software from Omni-Secure of Austin, TX, and AuthPro from CGI-City LLC of Columbus, OH, among others.

The system 200 may receive one or more default settings from the surgeon, as indicated at block 312. In an embodiment, the HMI entity 222 may provide a My Account screen or page for presentation to the surgeon. The My Account page may include fields or other data entry display elements for receiving one or more default settings. For example, the surgeon may specify a first default setting that identifies a particular type or brand of pain medication to be prescribed to a patient following a first surgical procedure. Similarly, the surgeon may specify a second default setting that identifies a different type or brand of pain medication and/or a different dosage to be prescribed following a second surgical procedure. The database management system 228 may construct an account record for the surgeon, and may save the specified default settings in the account record created for the surgeon, as indicated at block 314. The account record may be stored in the template database 226.

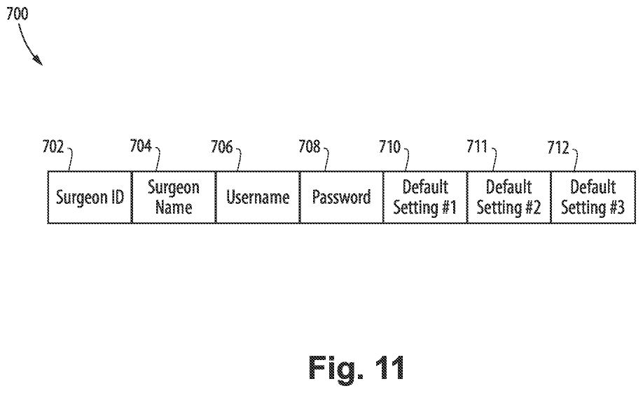

FIG. 11 is a schematic illustration of an account record 700 created by the database management system 228 for a surgeon, and stored in the template database 226. The account record 700 may include a surgeon identifier (ID) field 702, a surgeon name field 704, a username field 706, and password field 708. The surgeon ID field 702 may contain an identifier, such as a numeric or alphanumeric identifier assigned, e.g., by the database management system 228, to the surgeon. The surgeon name field 704 may contain the name of the surgeon, the username field 706 may contain a username, which may be selected by the surgeon. The password field 708 may contain a password, which also may be created by the surgeon. As mentioned, the username and password values may used by the surgeon to access the system 200. The account record 700 also may include one or more default setting fields, such as default setting fields 710-712. The default settings fields 710-712 may contain values corresponding to the default settings specified by the surgeon.

### System Operation

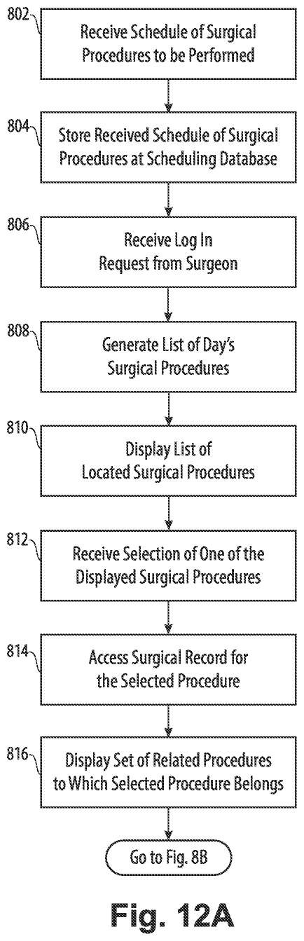

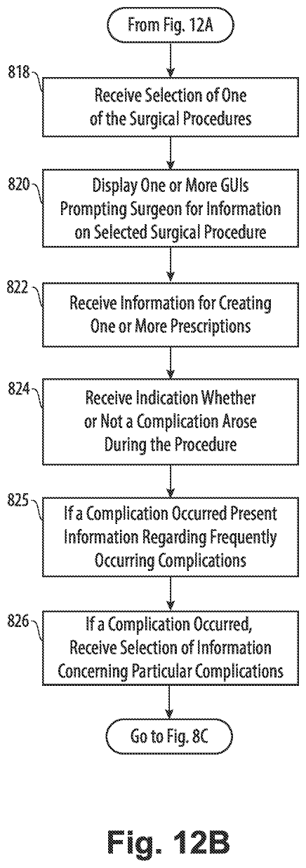

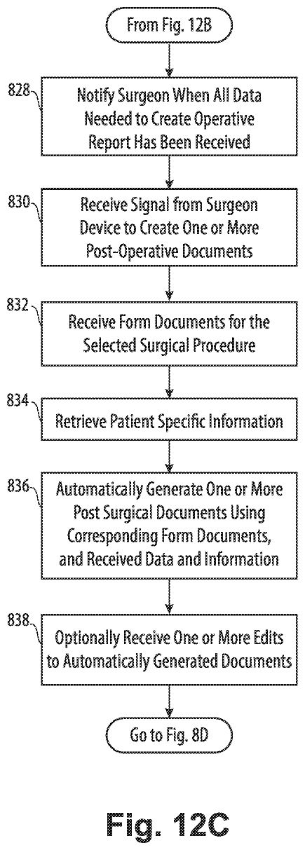

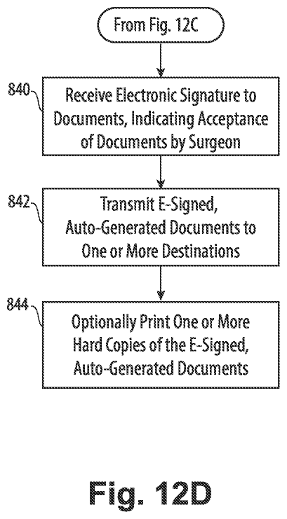

FIG. 12 is a flow diagram of a method in accordance with an embodiment of the present invention.

A user, such as a hospital administrator, may log into the document server 132, and enter or upload a schedule of surgical procedures that are to be performed, e.g., in the operating rooms 102-104 of the surgical suite 100, and the schedule may be received by the document server 132, as indicated at block 802. For each procedure, the administrator may enter the patient's name, date of birth, Medical Record Number (MRN), date and time of surgery, a general description of the type of surgical procedure, operating room, name of surgeon, and name of anesthesiologist. In an embodiment, hospital administrator may specify the names of the procedures in terms of the procedures associated with root nodes. That is, the procedures correspond to the procedures associated with root nodes, e.g., root node 408, of the hierarchical tree structures, e.g., tree 400.

It should be understood that additional or other information may be entered by the administrator. The database management system 228 may store the received scheduling information in the scheduling database 224, as indicated at block 804. The entry of information for scheduled procedures may be performed periodically, e.g., daily, weekly, etc. In addition, the hospital administrator may update the information as a result of schedule changes.

Suppose, for example, that the schedule includes a carpal tunnel release procedure that is to be performed on patient John White by Dr. John Rambo. The patient may be brought to an operating room, such as operating room 102, and the surgeon may perform the scheduled procedure on the patient. During the course of the surgical procedure one or more complications may arise, and those complications may be addressed by the surgeon. The surgical procedure may result in some blood loss by the patient, and the patient may be given intravenous fluids. The surgeon also may discover a mass or other object, and may take a specimen of the mass or object.

After completing a surgical procedure, the surgeon may log into the system 200 and cause a completed operative report to be generated instantly and automatically. The surgeon may also cause one or more other documents to be generated instantly and automatically. In an embodiment, the surgeon may utilize the touchscreen unit 116 located in operating room 102 to initiate the creation of the one or more post-operative documents, such as the operative report. That is, the surgeon may direct the system 200 to create one or more medical documents before he or she even leaves the operating room.

More specifically, the HMI entity 222 may present a log in screen, and the surgeon may log onto the server 132, for example by selecting his or her username and entering his or her password, as indicated at block 806. The server 132 may verify the entered credentials. Upon a successful login, the scheduling entity 212 may search the schedule database 224 for the surgical procedures scheduled for that day, as indicated at block 808. The HMI entity 222 in operation with the scheduling entity 212 may present the located procedures on the touchscreen unit 116, as indicated at block 810. In an embodiment, displayed procedures include all procedures scheduled for that day in the operating room, e.g., room 102, in which the touchscreen unit, i.e., unit 116 is located. For example, the document server 132 may receive an identification (ID) value assigned to the touchscreen unit 116, and thereby determine that the surgeon logged in through touchscreen unit 116 located in operating room 102. In response, the scheduling entity 212 may refine its search of procedures to operating room 102, and may present a list of the procedures scheduled in operating room 102. In another embodiment, the surgeon, after logging in to the system 200, may be presented with a list of operating rooms, and may select a particular operating room, e.g., room 102. In a further embodiment, the scheduling entity 212 may present a list of all of the procedures in any location that the logged in surgeon is scheduled to perform that day (or another time period).

Each entry in the list presented on the touchscreen unit 116 may include: patient name; patient date of birth, type of surgery; and time of surgery. If the list includes all procedures for the respective day, then each entry may also include the operating room number. It should be understood that additional and/or different information may be included in each entry of the presented list.

In an embodiment, the HMI entity 222 presents the list of surgical procedures in the form of a circular menu in the form of an analog clock face. Specifically, an image of a running clock having hour, minute, and second hands may be displayed, and the clock may be configured to present the current time. Surrounding the numbers on the clock face, e.g., the numbers 1-12, may be the surgical procedures. The procedures may be positioned around the clock face at or near their respective times. For example, the entry for a procedure scheduled for 10:00 a.m. may be located at the 10:00 a.m. position on the clock face, a procedure scheduled for 1:00 p.m. may be located at the one o'clock position of the clock face, and so on.

The surgeon may select the just-completed procedure from the list of procedures presented on the touchscreen unit 116, as indicated at block 812. It should be understood that selection may be accomplished in a variety of ways. For example, the selection of a desired entry may be made by touching with a fingertip that entry as displayed on the touchscreen unit 116. In response to the selection of a desired entry from the displayed list, the document constructor 214 may access the one or more records 700 stored in the template database 226 for the selected procedure, as indicated at block 814. If the selected procedure is associated with a root node, the document constructor 214 may access all of the L1 procedures that are associated with the root node of the selected procedure. For example, the selected procedure may be associated with a group ID, and the HMI entity 222 may locate all surgical procedure records 500 having that group ID value in their group ID fields 506.

The HMI entity 222 may generate a display on the touchscreen unit 116 of at least some, and preferably all, of the level one (L1) procedures, as indicated at block 816. For example, suppose the selected procedure is a carpal tunnel release. The HMI entity 222 displays at least some, and preferably all, of the procedures located at level one (L1) of the hierarchical tree 400 associated with carpal tunnel release.

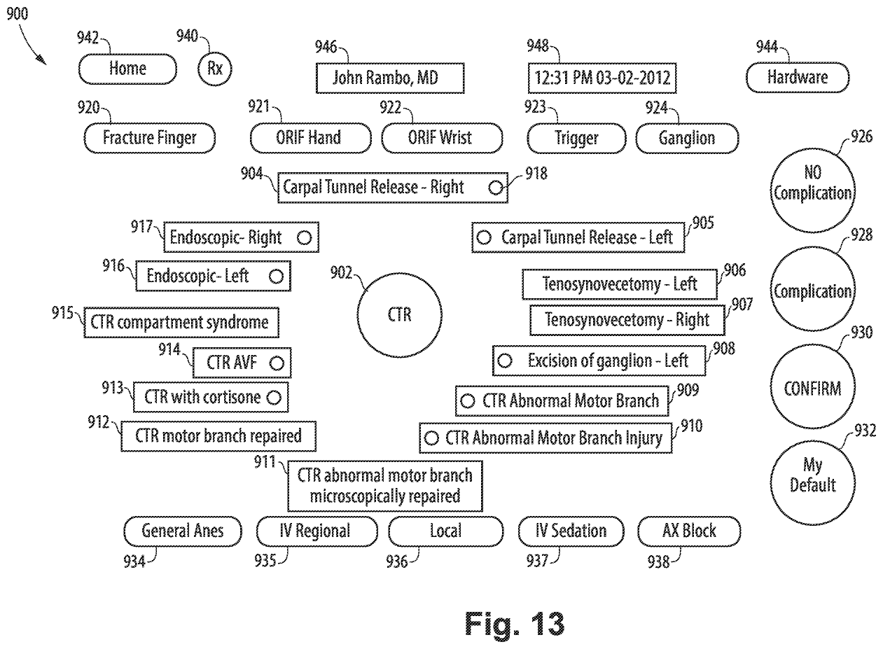

FIG. 13 is a schematic illustration of an exemplary surgical procedures graphical user interface (GUI) 900 constructed by the HMI entity 222 and presented on the touchscreen unit 116 to the surgeon. The GUI 900 includes a first display widget 902 that identifies the selected procedure, e.g., carpal tunnel release (CTR). Surrounding the first display widget 902 in a circular or ring type of arrangement may be a plurality of display items 904-917. Each display item 904-917 corresponds to one of the child surgical procedures for the parent procedure listed in the first display widget 902. To the extent a surgical procedure included in a display item, such as display item 904, has one or more child procedures associated with it, then the display item 904 may include a selectable feature, such as a command button 918, for accessing such one or more child surgical procedures. That is, in response to selection of the button 918, the procedure of display item 904 is moved to the first display widget 902, and the display items 904-917 display the child procedures for the selected procedure. In an embodiment, the procedures listed in display items 904-917 are presented in their rank order. For example, suppose the ring of display items 904-914 is a clock face with the first display widget 902 at the center. The most commonly performed procedure may be presented at the twelve o'clock (or one o'clock) position, while the least commonly performed procedure may be presented at the eleven o'clock (or twelve o'clock) position. Nonetheless, those skilled in the art will understand that other placement strategies may be used.

The surgical procedures GUI 900 also may include one or more surgical category buttons, such as a Fracture Finger surgical category button 920, an Open Reduction Internal Fixation (ORIF) surgical category button 921, an ORIF Wrist surgical category button 922, a Trigger surgical category button 923, and a Ganglion surgical category button 924. Each of the surgical category buttons 920-924 may present one or more other surgical categories that can be selected by the surgeon if the procedure that was performed on the patient does not match any of the procedures included in the display items 904-917. In an embodiment, one or more of the surgical category buttons 920-927 may be in the form of a spin wheel that can be operated by the surgeon to present further procedures for possible selection by the surgeon.

The surgical procedures GUI 900 also may include one or more command buttons, such as a No Complication button 926, a Complication button 928, a Confirm button 930, and a My Default button 932. In other embodiments, the No Complication button 926 may be omitted.

The surgical procedures GUI 900 may include one or more buttons for specifying an anesthesia. For example, the GUI 900 may include a General Anesthesia button 934, an Intravenous (IV) Regional button 935, a Local Anesthesia button 936, an IV Sedation button 937, and an Axiliary (AX) Block button 938. The surgeon may indicate the type of anesthesia that was used by selecting a corresponding one of the anesthesia buttons 934-938. For example, if a local anesthesia was used, the surgeon may select the local button 936. In response, the document constructor 214 may associate the selected anesthesia with the surgical procedure. In addition, predefined text describing the selected anesthesia, which may be stored in the template database 226, may be accessed for use in constructing the operative report and/or one or more other post-operative documents. In an embodiment, upon selecting one of the anesthesia buttons 934-938, the HMI entity 222 may present one or more other screens on the touch screen unit 116 through which the surgeon may select one or more details of the respective anesthesia, such as the amount used, etc.

The GUI 900 also may include a prescription (Rx) button 940, a Home button 942, and a Hardware button 944. If the surgeon selects the Rx button 940, the HMI entity may present one or more screens on the touch screen unit 116 for receiving selections of one or more prescriptions to be prescribed to the patient. If the surgeon selects the Home button 942, the document constructor 214 may return to a screen presenting a plurality of surgical procedures. If the surgeon selects the Hardware button 944, the HMI entity 222 may present one or more screens for receiving information concerning the surgical hardware that was used during the procedure, such as the number and type of surgical screws, the number and type of plates, etc.

The surgical procedures GUI 900 may include a surgeon name element 946 that displays the name of the surgeon operating the system 200, and a date/time element 948 that displays the current date and time.

The surgical procedures GUI 900 is meant for illustrative purposes and the present invention may be utilized with other GUIs having greater or fewer graphical elements.

If the procedure performed on the patient is one of the procedures displayed in one of the display items 904-917, such as carpal tunnel release-right, then the surgeon may select that procedure by selecting the display item, i.e., display item 904, as presented on the touchscreen unit 116, as indicated at block 818 (FIG. 8B). The surgeon may use a touch gesture to select the display item 804.

In another embodiment, the HMI entity 222 may be configured to present one or more graphical images for use in selected the procedure that was performed. For example, the HMI entity 222 may present a graphic image of all or a portion of the human body. The surgeon may select the area that was operated on by touching that portion of the human body image as displayed on the touch screen unit 116. The surgeon may zoom in or out of the graphical image, for example using finger pinch gestures, or finger tap gestures, etc. In response, the HMI entity 222 may present one or more close-up images of the selected portion of the human body to facilitate the identification of the location at which the surgery that was performed. For example, the surgeon may first select, e.g., by touching, the right hand of the image of the body. The HMI entity 222 may present a graphic image of a hand. The surgeon may then select, e.g., by touching, the index finger of the graphic image of the hand. The HMI entity 222 may present a list of the surgical procedures that may be performed on the right index finger, and the surgeon may select from this list the procedure that was performed on the patient. In this embodiment, the template database 226 may include a mapping between portions of a body, and the surgical procedures that may be performed on that portion of the body.

In an embodiment, the one or more graphical images may include images of internal anatomical structures, such as the heart, lungs, stomach, etc., and surgical procedures relating to these structures or organs may be displayed and selected.

The information or data entered by the surgeon concerning the surgical procedure may be saved by the document constructor 214. In an embodiment, the document constructor 214 may save the information or data in main memory. Alternatively, the document constructor 214 may save the information or data in the template database 226.

In response, the HMI entity 222 may present one or more other GUIs that prompt the surgeon for information concerning the procedure that was performed, as indicated at block 820. For example, the HMI entity 222 may present one or more other GUIs on the touchscreen unit 116 that prompt the surgeon, for example, to enter the estimated blood lost by the patient during the procedure, e.g., in milliliters (ml), the amount of intravenous fluids given to the patient in ml, the identity of any specimens that were taken from the patient, whether any cultures were obtained and sent for analysis, the identity of any drains that were placed in the surgical site, the use of any splints, and the type of skin closure that was used. It should be understood that depending on the procedure that was performed, other information or data may be obtained from the surgeon.

Information for presenting additional GUIs may be obtained from respective fields of the surgical procedure record 500 for the selected surgical procedure.

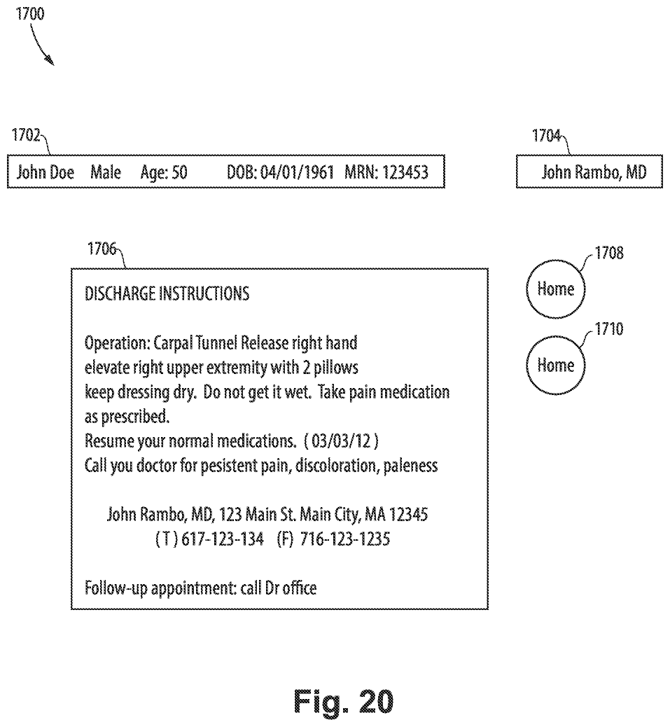

FIG. 20 is a schematic illustration of a skin closure GUI 1600 that may be created by the HMI entity 222 and presented on the touchscreen unit 116 to the surgeon. The skin closure GUI 1600 may have a patient data element 1602 that includes information about the patient for whom the operative report is being created, e.g., John White whose date of birth is Jan. 7, 1956. The skin closure GUI 1600 also may include a surgeon data element 1604 that includes the name of the surgeon utilizing the system 200, e.g., John Rambo, MD, and a date/time element 1606 that presents the current date and time.

In an embodiment, the skin closure GUI 1600 may include a skin closure selection area 1608. The skin closure selection area 1608 may include a plurality of buttons corresponding to the types of skin closures that might be performed for the particular surgical procedure. The buttons, moreover, may be arranged in the form of a clock face that circle around a central display element 1610. For example, starting at the 12 o'clock position, the particular skin closure buttons may include an Interrupted Nylon button 1612, an Interrupted Prolene button 1613, a Subcuticular Pull Out button 1614, a Subcuticular Absorbable button 1615, a Layered Closure button 1616, a Layered Absorbable button 1617, a Layered Non-Absorbable button 1618, a Mattress Closures button 1619, a Not Closed button 1620, a Partially Closed button 1621, a Closed with Drains button 1622, a Staple Closure button 1623, a Stay Sutures button 1624, a Half Buried Horizontal button 1625, a Steel Wire Closure button 1626, and a Vac Dressing button 1628. These predefined types of skin closures may be obtained from the skin closure field 530 of the respective surgical procedure record 500.

The surgeon may select the button that corresponds to the type of closure that was actually performed during the procedure, e.g., the Staple Closure button 1623. In response, the document constructor 214 may associate a predefined description of that type of skin closure with the surgical procedure.

The skin closure GUI 1600 also may include a Confirm button 1630, and a Prescription (Rx) button 1632. After choosing the button corresponding to the skin closure that was used, e.g., the Staple Closure button 1623, the surgeon may select the Confirm button 1630.

The HMI entity 222 may present other GUIs to obtain information from the surgeon regarding the surgical procedure. For example, the HMI entity 222 may be configured to present a findings GUI that presents a plurality of findings associated with the particular surgical procedure that was performed, as obtained from the findings field 534. For example, for the findings associated with the appendectomy surgical procedure may include Normal Appendix, Appendicitis, Necrosis, Purulent, Ilcoccal Mass, Appendix with CA, Small Bowel Perforation, Lymphadenitis, Crohn's Disease, PID, Sigmoid Diverticulitis, Etopic Pregnancy, and Ovarian Torsion. The available findings may be presented as buttons in a clock face arrangement, and the surgeon may the select the button that corresponds to the finding that occurred during the surgical procedure. In response, the document constructor 214 may retrieve the predefined text associated with the selected findings, and include the predefined text in the operative report or other document being generated.

The document constructor 214 also may receive information for automatically creating one or more prescriptions for the patient, as indicated at block 822. In an embodiment, the HMI entity 222 may generate and present a prescription GUI on the touchscreen unit 116 prompting the surgeon for, and receiving, prescription related information. The surgeon may access the prescription GUI by selecting the Rx button 940 from the GUI 900.

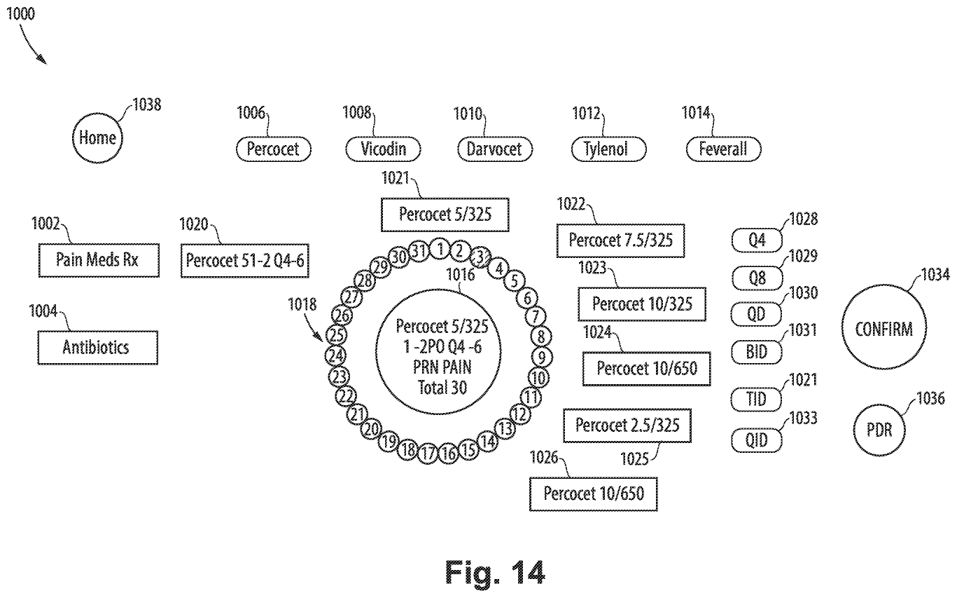

FIG. 14 is a schematic illustration of a prescription data collection GUI 1000 that may be presented on the touchscreen unit 116 in accordance with an embodiment of the present invention. The prescription GUI 1000 may include one or more selectable buttons or tabs for selecting a particular category of medication to be prescribed, such as a Pain Medication (Meds) prescription (Rx) category button or tab 1002 and an Antibiotics category button or tab 1004. The GUI 1000 of FIG. 14 illustrates the pain medication tab 1002. That is, the system 200 presents the GUI 1000 in response to receiving a selection of the Pain Meds Rx button or tab 1002 by the surgeon. In an embodiment, the document constructor 214 in operation with the HMI entity 222 presents on the prescription GUI 1000 the most commonly prescribed pain medications for the particular type of surgery, which may be stored in the medication fields 524, 526 of the respective surgical procedure record 500.

More specifically, the GUI 1000 may include a plurality of selectable buttons corresponding to the most commonly prescribed pain medications, such as a first button 1006 for Percocet, a second button 1008 for vicodin, a third button 1010 for darvocet, a fourth button 1012 for Tylenol, and a fifth button 1014 for feverall. The GUI 1000 further includes a current selection display element 1016 that displays the currently selected pain medication, the currently selected dosage and the currently selected number of days. The GUI 1000 further includes a days of the month element 1018 that includes a separate button for each day in a month, e.g., 1 through 31. In an embodiment, each number of days button is a selectable button, and the set of buttons 1018 are arranged in the form of a ring around the current selection element 1016. The number of days buttons 1018 (1-31) may be configured to have the appearance of a clock face with the current selection element 1016 in the center of the clock face. The prescription data collection GUI 1000 also may include a Home button 1038.

In response to receiving the selection of a particular pain medication, e.g., via selection of one of the buttons 1006-1014, such as the Percocet button 1006, the HMI entity 222 may present one or more selectable buttons corresponding to different strengths of that medication that are most commonly prescribed. In particular, the GUI 1000 may include strength buttons 1020-1026 for the pain medication Percocet. The GUI 1000 also may include one or more selectable dosage buttons, such as dosage buttons 1028-1033. Each selectable dosage button 1028-1033 may correspond to a different dosage, such as once every four hours (Q4), once every eight hours (Q8), four pills a day (QD), twice a day (BID), three times a day (TID), and four times a day (QID).

The document constructor 214 may receive the following information as entered by the surgeon in the GUI 1000: type of pain medication, e.g., Percocet, strength, e.g., 5/325, number of days, e.g., three, and frequency, e.g., once every four hours (Q4). Based on this information, the document constructor 214 may compute the total number of pills needed to meet the entered prescription, e.g., 30. The HMI entity 222 may present the received selections and the computed total number of pills in the current selection display element 1016 for review by the surgeon. If correct, the surgeon may indicate his or her approval by selecting a Confirm command button 1034 of the GUI 1000. The document constructor 214 may save the received information in the template database 226.

In an embodiment, the prescription GUI 1000 also may include a Physician Desk Reference (PDR) button 1036. The surgeon may select the PDR button 1036 to obtain information regarding a particular medication. More specifically, in response to the selection of the PDR button 1036, the document server 132 may access the drug information database 206 via interface 232. For example, the document server 132 may issue one or more commands, such as one or more Application Programming Interface (API) commands, Remote Procedure Calls (RPCs), etc., through the interface 232 to the drug information database 206 to obtain drug information. The drug information database 206 may represent a repository of information regarding medications and drugs, such as their common uses, possible side effects, interactions with other medications, etc.

The HMI entity 222 may present other GUIs for prompting the surgeon to enter, and for receiving, information for one or more other prescriptions, such as a prescription for an antibiotic.

The system 200 also may receive an indication from the surgeon of whether or not a complication occurred during the procedure, as indicated at block 824 (FIG. 12B). For example, if the procedure took place without any complications, the surgeon may select the No Complication button 926 (FIG. 13). If a complication arose, the surgeon may provide information concerning the complication by selecting the Complication button 928. The HMI entity 222, in response to the surgeon's selection of the Complication button 928, may access the one or more common complications as specified in the complications field 518 of the surgical procedure record 500. The HMI entity 222 may present the retrieved complications, and present them to the surgeon on the touchscreen unit 116, as indicated at block 825. The surgeon may select, from the list presented on the touchscreen unit 116, the complication that occurred during the surgical procedure, thereby providing information concerning the complication to the document server 132, as indicated at block 826. The document constructor 214, in response to the surgeon selecting one of the complications, retrieves a predefined description of that complication from the template database, and automatically adds this description to the operative report being constructed.

In an embodiment, the document constructor 214 notifies the surgeon when all of the data required to generate the operative report or other medical document has been received, as indicated at block 828 (FIG. 12C). For example, the document constructor 214 operating with the HMI 222 may change the appearance of the confirm button 930, e.g., from the color red to the color green. In an embodiment, the document constructor 214 determines that all necessary information has been received when data for each of the placeholders in the form document, e.g., form 600, has been received. The document constructor 214 may receive a signal to create one or more documents for the surgical procedure, as indicated at block 830. For example, once the confirm button 930 has been transitioned from red to green, it may be selected, e.g., touched, by the surgeon.

In response, the document constructor 214 may retrieve the one or more form documents for the particular type of surgical procedure that was selected by the surgeon, as indicated at block 832. For example, the document constructor 214 may utilize the type of procedure specified on the surgical procedure GUI 800 to index into the database of surgical records, and identify the one or more surgical records 500 corresponding to the procedure specified by the surgeon. The document constructor 214 also may retrieve patient-specific information for the patient on whom the procedure was performed, as indicated at block 834. The patient-specific information may be stored in the scheduling database 224 at the record for the selected procedure. Alternatively, the document constructor 214 may issue one or more commands, such as one or more Application Programming Interface (API) commands, Remote Procedure Calls (RPCs), etc., via the interface 233 to the patient record system 208 to obtain patient-specific information.

Utilizing the information or data specified by the surgeon, and the retrieved patient-specific information, the document constructor 214 may automatically create one or more documents, as indicated at block 836. For example, the document constructor 214 may automatically generate an operative report, a set of recovery room instructions, discharge instructions, and one or more prescriptions.

The document constructor 214 may generate these documents in completed form without any further input from the surgeon. For example, the operative report, as generated by the document constructor 214, may include patient name, date of birth, sex, pre-operative diagnosis, date of surgical procedure, surgical code, a description of the surgical procedure as performed, the name of the anesthesiologist, the estimated blood loss, the volume of intravenous fluids administered to the patient, complication, type of skin closure performed, the identity of any specimens taken from the patient, the identity of any cultures obtained, e.g., for microbiology analysis, the placement of any drains, etc.

The operative report also includes the appropriate codes, e.g., CPT and/or ICD codes for the procedure(s) that were performed. The codes may be obtained from the CPT and ICD fields 520, 522 of the surgical record 500 for the procedure that was performed. That is, the document constructor 214 automatically enters the appropriate CPT and/or ICD codes into the operative report being generated, thereby eliminating the need for the surgeon to know or look-up the proper codes, or to manually enter them into the operative report.

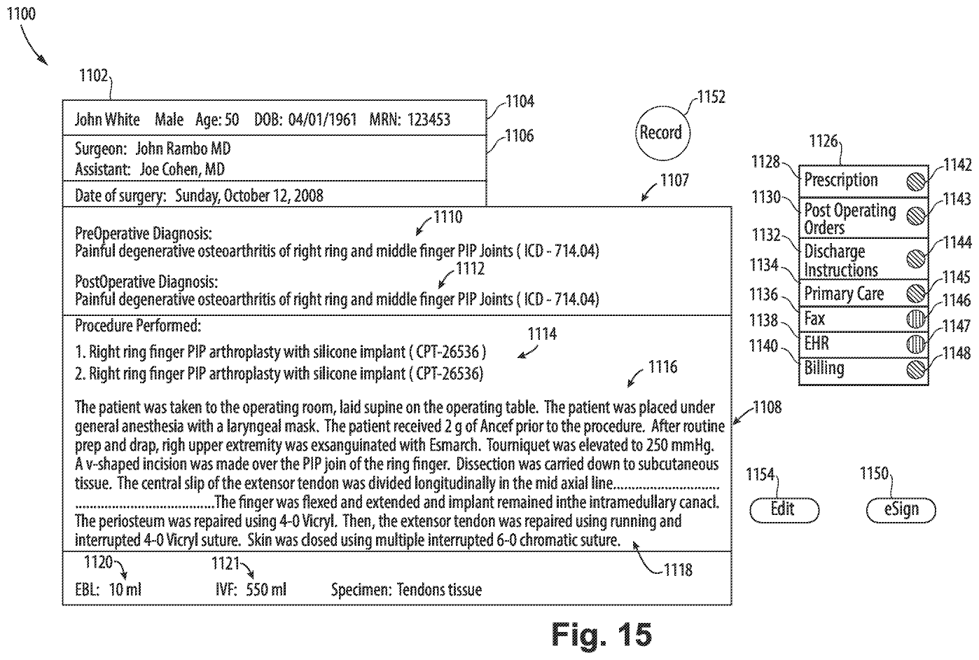

FIG. 15 is an illustration of an operative report GUI 1100 presented on the touchscreen unit 116 to the surgeon. The operative report GUI 1100 includes at least a portion of an operative report 1102 completed automatically by the document constructor 214. The operative report 1102 includes one or more patient-specific information elements, such as element 1104 that contains the patient's name, sex, date of birth, and medical record number (MRN). As discussed, the document constructor 214 obtained the patient-specific information from the scheduling database 224 and/or the patient record system 208. The operative report 1102 also includes one or more schedule-based elements, such as schedule-based element 1106, which may include the name of the surgeon, the name of the surgeon's assistant, the name of the anesthesiologist, and the location where the surgery was performed. As discussed, information for element 1106 may have been received by the document constructor 214 from the scheduling database 224. The operative report 1102 may include diagnosis portion 1107, and a body portion 1108. The diagnosis portion 1107 may include a pre-operative diagnosis entry 1110, and a post-operative diagnosis entry 1112. The body portion 1108 may include a summary 20) description summary of the procedure entry 1114, and a detailed description of the procedure entry 1116. The operative report 1102 portion may include elements that correspond to the data obtained from the surgeon through the touchscreen unit 116. For example, the detailed description section 1116 may include a first element 1118 describing the skin closure used during the procedure. The operative report 1102 also may include an estimated blood loss (EBL) entry 1120 that contains the value, e.g., 10 ml, as entered by the surgeon, a intravenous fluid (IVF) entry 1121 that contains the value, e.g., 550 ml, as entered by the surgeon, and a specimen entry 1122 that contains the description of the specimen taken, e.g., tendons tissue.

The operative report GUI 1100 also may include a status window 1126. The status window 1126 may include one or more display elements for tasks associated with the operative report. For example, the status window 1126 may include a prescription element 1128, a Post Operative Orders element 1130, a Discharge Instructions element 1132, a Primary Care element 1134, a facsimile (Fax) element 1136, an EHR element 1138, and a Billing element 1140. Each element 1128-1140 may include a corresponding status indicator, such as status lights 1142-1148, respectively. The HMI entity 222 may set the status indicator based on the current status of the corresponding task. For example, the HMI entity 222 may set a status indicator to the color red to indicate an incomplete task, and to the color green to indicate a completed task. As illustrated in FIG. 15, only the Send element 1134 remains incomplete.

The operative report GUI 1100 may also include one or more command buttons that may be selected by the surgeon. Specifically, the GUI 1100 may include an electronic signature (eSign) button 1150, and a Record button 1152. When the surgeon is satisfied with the auto-generated operative report 1102, he or she may select the eSign button 1150, e.g., with a touch gesture.

The surgeon may, if necessary or desired, edit, modify or revise the automatically generated operative report 1102, as indicated at block 838 (FIG. 12C). For example, the operative report GUI 1100 also may include an Edit command button 1154. If the surgeon selects the Edit command button 1154, the document constructor 214 may permit the surgeon to edit some or all of the operative report as presented on the operative report GUI 1100.

The surgeon may append information to the auto-generated operative report 1102 by selecting the Record button 1152 and verbally describing the additional information while facing the touchscreen unit 116. Selecting the Record button 1152 may start a the video recording feature of the touchscreen unit 116, which may be configured to display back to the surgeon the video data as it is being recorded. When the surgeon has finished describing the additional information he or she may again select the Record button 1152, which may be converted to a Stop button during the recording process. The video recording, which may be in the form of a .wav or other media file, may be appended to one or more of the documents being generated, such as the operative report. In an embodiment, the video recording may be transmitted to a transcription service, and a transcription of the audio portion of the video recording may be created. This transcription report may be appended to one or more of the documents, such as the operative report.

FIG. 21 is a schematic illustration of a discharge instructions GUI 1700 that may be created by the HMI entity 222, and presented to the surgeon on the touch screen unit 118. The discharge instructions GUI 1700 may be based on predefined information in the form discharge instructions field 514 of the respective surgical procedure record 500, and may include a patient information element 1702 that may display information about the patient, such as name, sex, age, date of birth DOB), and medical record number (MRN). The discharge instructions GUI 1700 also may include a surgeon information element 1704 that may display the name of the surgeon. The discharge instructions GUI 1700 also may include a discharge instructions element 1706 that may display the discharge instructions associated with, e.g., assigned to, the particular surgical procedure that was performed. The discharge instructions may be stored in the template database 226. The discharge instructions GUI 1700 also may include a Home button 1708, and a Print button 1710. The surgeon may cause the discharge instructions as shown in the discharge instructions element 1706 to be printed, e.g., on printer 120, by selecting the Print button 1710, e.g., with a finger gesture, such as tapping the Print button 1710.

The surgeon may accept the auto-generated documents, e.g., operative report, prescription(s), discharge and/or recovery room instructions, post-operative orders, post-operative instructions, etc., by applying his or her electronic signature (e-signature) to the documents, which is received by the system as indicated at block 840 (FIG. 12D). The document distribution entity 216 may transmit the signed, auto-generated documents to one or more predetermined locations or destinations, as indicated at block 842. For example, the operative report, recovery room instructions, and discharge instructions may be sent to the hospital's patient record system, while the prescription may be sent to the hospital pharmacy or faxed to the patient's preferred pharmacy. One or more of the documents may be transmitted as part of an email to one or more email addresses. One or more of the documents may be faxed to one or more facsimile numbers. The operative report and/or other automatically generated documents may be transmitted to one or more health insurance companies as part of a claim.

In an embodiment, the document server 132 may be individually configured for each installation. For example, at a first healthcare facility, the document server 132 may be configured to email reports to a predetermined set of recipients. At a second healthcare facility, the document server 132 may be configured to fax reports to a predetermined fax number.

In addition, the document distribution entity 216 may print hard copies of one or more of the auto-generated documents, as indicated at block 844. The hard copies may be added to the patient's physical file that may be transported with the patient as he or she is moved from the operating room to the recovery room 106. For example, one or more of the documents may be printed by printer 120 in the third operating room 104 and/or by printer 122 in the recovery room 106.

In an embodiment, the surgeon may access the document server 132 from other devices besides the touchscreen units 116-118 in the operating rooms 102-104. For example, the surgeon may use a mobile device, a desktop device, or other computing platform to access the document server, and initiate the generation of the documents. A surgeon may utilize a desktop computer, such as the desktop computer 124 located in his or her office 108. A surgeon also may use a mobile device to access the system 200 from within the surgeon's lounge 112.

As described, post-operative documents may be automatically created by the system 200 without any dictation being performed by the surgeon. Furthermore, the documents may be created with just a few touches on a touchscreen unit. In addition, the documents may be created by the system 200 without the surgeon having to enter data or make selections through a keyboard, a pointing device, a stylus, or pen and paper.

The GUIs created by the HMI entity 222 may be hyper-text markup language (HTML) or other web-based files, and may be presented, e.g., as one or more web pages, through a browser application running on a data processing device being operated by the surgeon.

### System Training/Learning

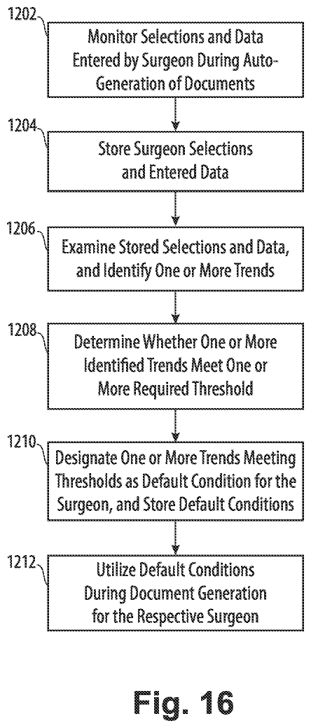

FIG. 16 is a flow diagram of a method in accordance with another embodiment of the present invention for training the system based on its use by individual surgeons. The training or learning module 236 of the document server 132 may monitor the use of the system 200 by one or more, and preferably all, of the surgeons, as indicated at block 1202. Specifically, the training module 236 may be configured to monitor the various selections made by the surgeons the first and each time they operate the system. This information may be appended to the surgeon's account record 700. In an embodiment, when a surgeon accesses the system to generate documents for a procedure that the surgeon has generated documents at least once before, the surgeon may select the My Default button 932 (FIG. 13). In response, the document constructor 214 may create the same set of documents as created the first time the surgeon used the system to generate documents for the procedure. The document constructor 214 and HMI entity 222 may present on the touchscreen unit 116 being accessed by the surgeon a completed operative report. That is, the document constructor 214 and the HMI entity 222 may transition directly from the surgical procedures graphical user interface (GUI) 900 directly to the operative report GUI 1100 (FIG. 15). The surgeon may then esign the operative report as presented on the touchscreen unit, and be finished with the process of creating the desired medical documents.

In a further embodiment, the training module 236 may monitor the selections made by the surgeons each time they operate the system. The training module 236 may store this data in the surgeon's account record 700 and/or the template database 226, as indicated at block 1204. The training module 236 may be further configured to examine the collected data, analyze the collected data, and identify one or more trends in the use of the system 200 by each surgeon, as indicated at block 1206. For example, the training module 216 may determine that the first surgeon prescribes the same type and dosage of pain medication following a particular procedure performed by the surgeon, e.g., carpal tunnel release. The training module 236 may determine whether the identified trends satisfy one or more thresholds or other criteria, as indicated at block 1208. For example, the training module 236 may first determine whether a given surgeon has made the same selection for a given surgical procedure at least 85% of the times, and that the given surgeon has used the system to generate documents for the given procedure at least some minimum number of times, e.g., ten. If a trend meets the one or more thresholds or criteria, the training module 236 may designate such a trend as a default condition for the first surgeon, as indicated at block 1210. If so, the training module 216 may store the trend as a default condition for the respective surgeon, as also indicated at block 1210.

Thereafter, in response to the first surgeon selecting the My Defaults button, instead of prompting the surgeon for information regarding a completed procedure, the document constructor 214 may retrieve the default condition, and utilize the retrieved default condition in the generation of one or more of the medical documents, as indicated at block 1212. Accordingly, the retrieved trend data as determined by the training module 236 may be used to complete the one or more medical documents automatically created for the surgical procedure.

### Customized Document Creation

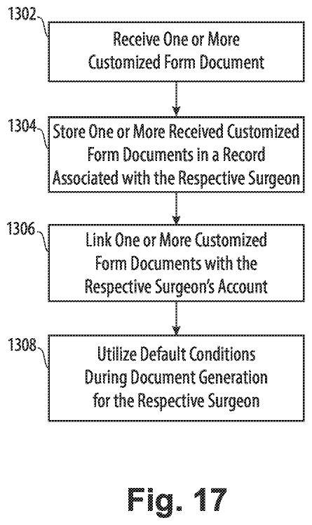

FIG. 17 is a flow diagram of a method in accordance with an embodiment of the present invention. In this embodiment, one or more surgeons may create one or more customized document templates for his or her practice. Specifically, a surgeon may create a customized operative report form for one or more surgical procedures that he or she performs. A surgeon may choose to create customized operative report forms only for those procedures that he or she regularly performs. Each customized operative report form may include a pre-operative diagnosis, a description of the procedure, one or more typical findings, and a post-operative diagnosis. The surgeon may create the customized form using a word processing application, such as Microsoft Word, and may save the customized form as a file. The surgeon may then log into the system 200, and upload the file to the document server 132, as indicated at block 1302. The database management system 228 may store the uploaded file in the template database 226, as indicated at block 1304. The database management system 228 may establish within the template database 226 one or more directories or folders associated with, e.g., assigned exclusively to, the surgeon, and may store the uploaded customized forms at these directories or folders. The database management system 228 may link the uploaded forms to the surgeon's account, as indicated at block 1306. For example, the database management system 228 may append the customized templates (or links to the customized templates) to the surgeon's account record 700.

The surgeon may also create other customized form documents in addition to the operative report. For example, for one or more of the surgical procedures, the surgeon may create postoperative care instructions, discharge instructions, etc. These additional customized forms may also be uploaded to the document server 132, and stored in the template database 226.

It should be understood that the format, language, organization, and content of the customized forms, as well as the specified preferences, may be custom tailored to the particular surgeon's practice.

In an embodiment, the HMI entity 222 may be configured to present one or more Graphical User Interfaces (GUIs) for receiving the customized forms, and preferences from the surgeon.

The database management system 228 may save the uploaded forms and the specified preferences in the template database 226.

As mentioned, the customized forms and the received preferences may be stored in directories or files assigned to the surgeon that uploaded the forms and specified the procedures. For example, the one or more customized forms can be included in the surgeon's account record 700.

In response, the document constructor 214 may access the template database 226, and retrieve the one or more customized forms, and the one or more preferences that the surgeon previously established for the type of procedure that was just performed when creating medical documents for the surgeon, as indicated at block 1308.

### Surgical Dashboard

In an embodiment, the HMI entity 222 in operation with the scheduling entity 212 may provide an operating room dashboard. The dashboard may present a list of all of the procedures being performed in the hospital's operating rooms on the current day, which is kept up to date throughout the day. The dashboard may be presented on the display unit 130 located in a surgeon's lounge 112. Nonetheless, the dashboard may be presented in alternative or additional locations.

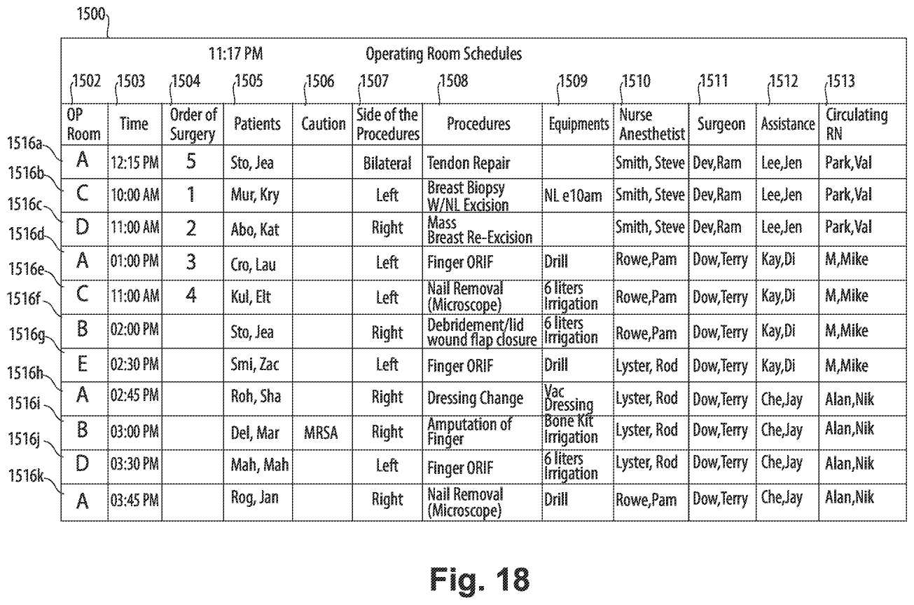

FIG. 18 is a schematic illustration of a dashboard 1500 as generated by the HMI entity 222 and presented on the display unit 130. The dashboard 1500 may include information indicating when a procedure started, and when it was finished, and this information may be continuously updated. By reviewing the information presented on the dashboard 1500, a surgeon can quickly determine whether a particular procedure is running behind schedule, thus delaying another procedure that the surgeon is scheduled to perform in that same operating room.

The operating room dashboard 1500 may also be accessed from other devices, such as mobile and desktop devices. For example, a surgeon may access the dashboard 1500 from the desktop computer 124 in his office 108. The surgeon may also access the dashboard 1500 from a mobile device, such as a smart phone or a tablet computer.

The dashboard 1500 may be organized as a table or array having a plurality of columns and rows. For example, the dashboard 1500 may include an Operating Room column 1502, a time of surgery column 1503, an order of surgery 1504, a Patient name column 1505, a Caution column 1506, a side of procedure column 1507, a procedure description column 1508, an equipments column 1509, a nurse anesthesiologist name column 1510, a surgeon name column 1511, an assistant name column 1512, and a circulating registered nurse (RN) name column 1513. The dashboard 1500 also may include a plurality of rows, such as rows 1516a-k, where each row 1516 may correspond to a particular procedure performed on a particular patient. The cells defined by the intersections of the columns and rows contain data for the respective procedure as defined by the column. For example, row 1516d identifies a procedure that will occur in operating room A, at 1:00 in the afternoon, with an order of surgery of 3. The name of the patient being operating on is Lau Cro. There is no caution. The patient is being operated on the left side. The procedure is a finger open reduction internal fixation (ORIF). The equipment to be used is a drill. The name of the nurse anesthesiologist is Pam Rowe. The name of the surgeon is Terry Dow, MD. The name of the assistant is Di Kay. The name of the circulating nurse is Mike M.

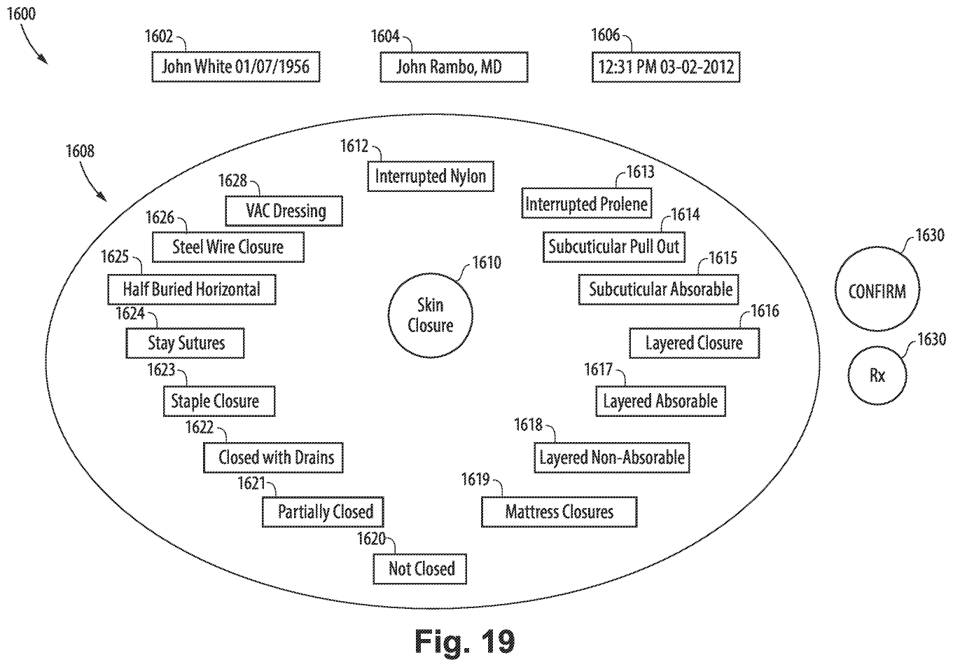

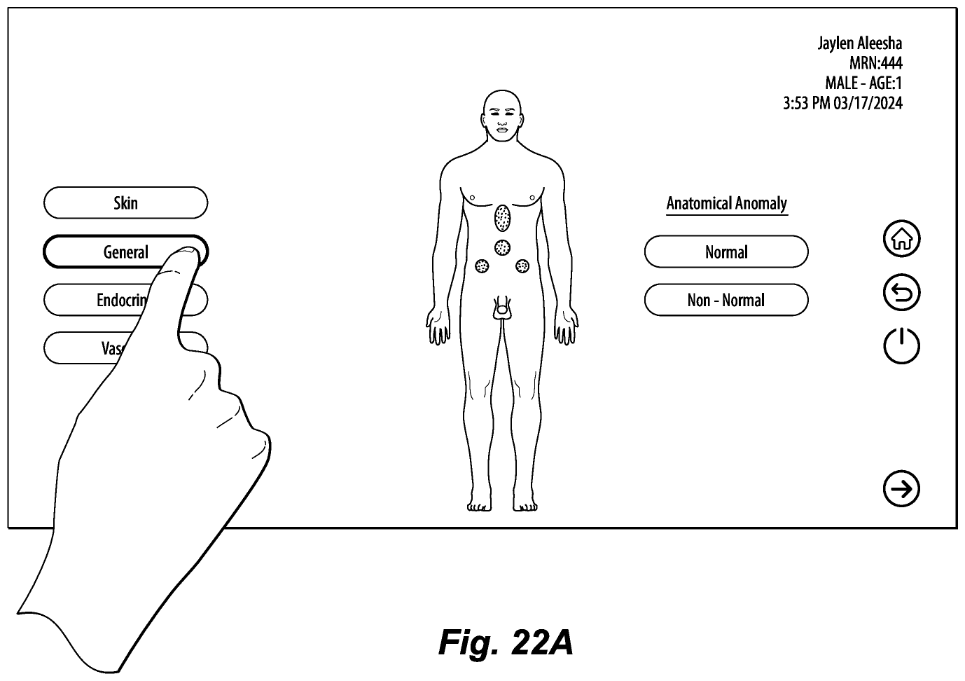

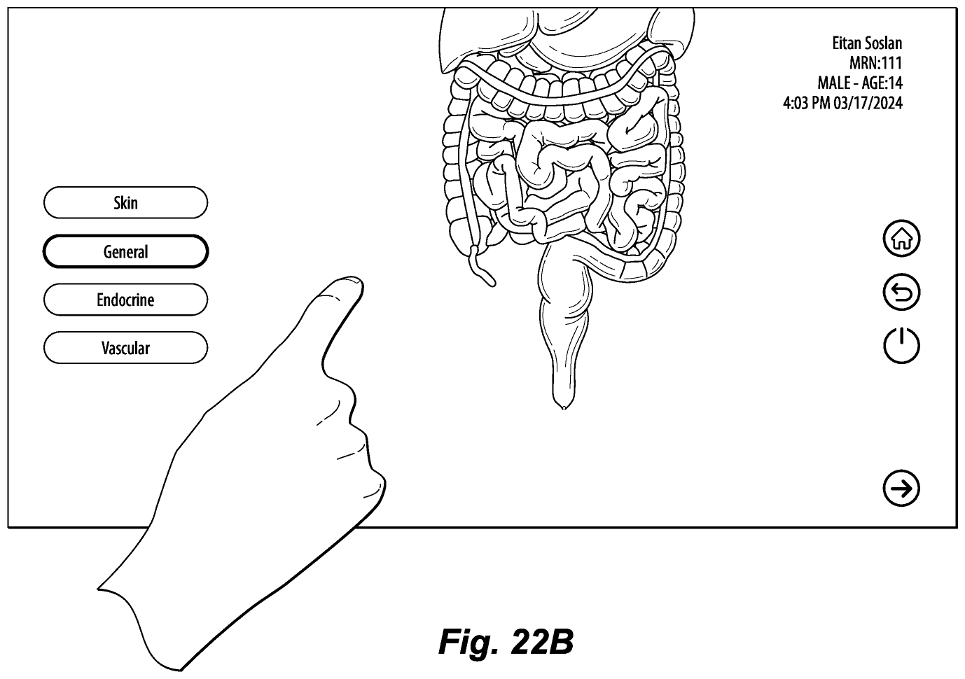

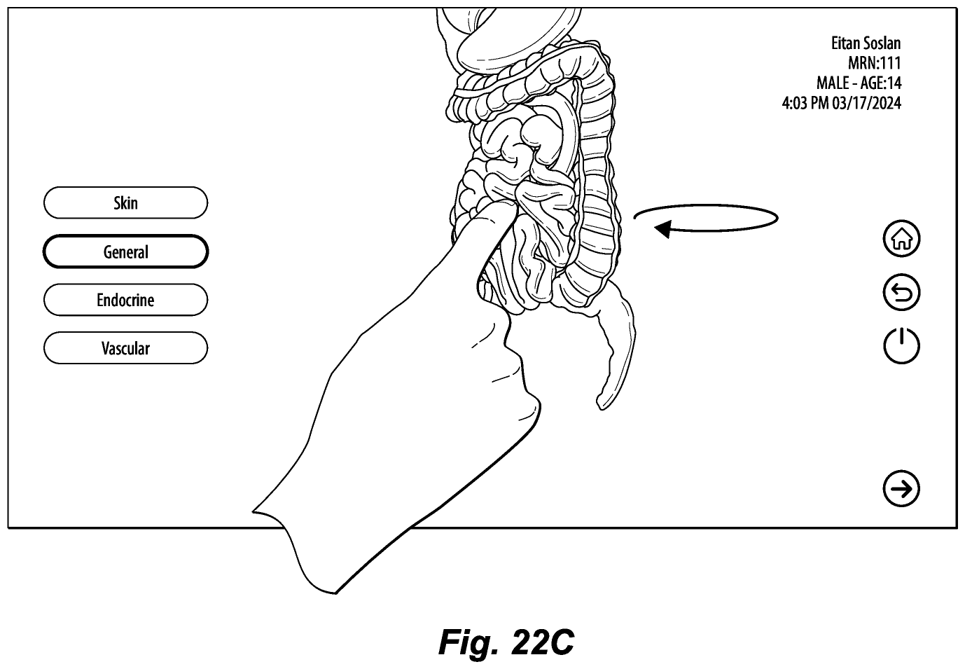

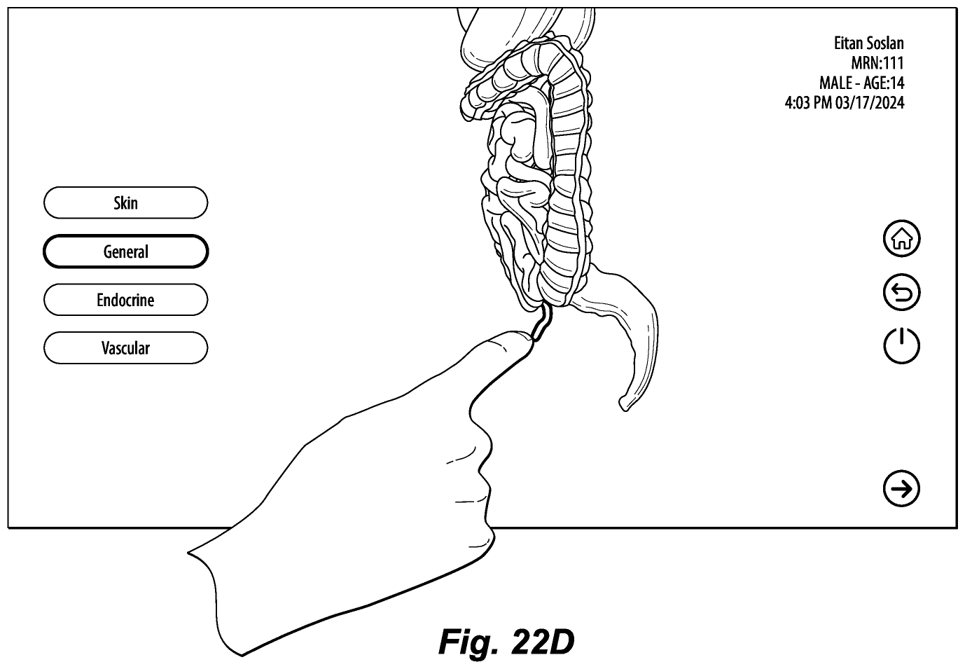

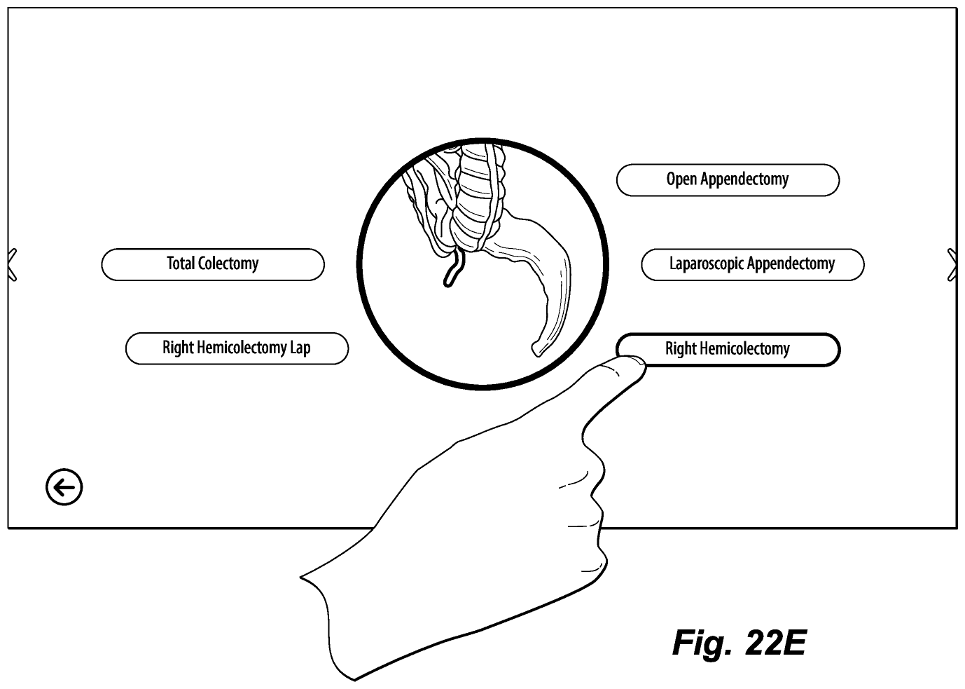

With reference now to the process 5000 of FIG. 21, and the exemplary illustrations of FIGS. 22A-22E depicting various GUI's generated on preferably a portable computing device 116 having a touchscreen interface (e.g., a smart tablet and/or phone device as described above). It is to be appreciated that process 5000 is performed by system 132 (e.g., a cloud-based system 200) as described above in accordance with one or more of the illustrated embodiments of FIGS. 1-20.

Starting at step S100, caused to be generated by computer system 132, on a computer display provided on user device 116, is a Graphical User Interface (GUI) that includes a user interactive animated visual representation of at least a portion of a human body 6000 representative of the patient (FIG. 22A) in conjunction with user touch interactive touch points (6002-6008) and identifiers (6010-6016) provided on the GUI.

As described above, the computer display is preferably a touch screen device wherein the user input consists of detection of user touch (6018) on the GUI. As also described above, the user device 116 receives, via a communications network, instructions and data from a remotely located computer server system 132 (e.g., a cloud-based computer system 200) for enabling operation of the GUI. Preferably, the interactive animated visual representation of at least a portion of the human body 6000 representative of the patient is a three-dimensional (3D) model of the human body portion 6000 that has an adjustable view perspective on the GUI via manipulation of the 3D human body model caused by user interactive touch input upon the GUI.

In accordance with certain illustrated embodiments, the adjustable view perspective of the 3D human body portion displayed on the GUI is caused by human touch upon the GUI of device 116, via user manipulation of the 3D human body model (as best shown in FIGS. 22B-E). In certain illustrated embodiments, the computer system 200 is operative and configured to determine, and generate for user selection on the GUI of device 116, a plurality of selectable anatomical anomalies that may be relevant to the patient (See, FIG. 22A). It is to be appreciated and understood that anatomical anomalies are normal body structures with different morphological features than those described in anatomy textbooks. They usually don't affect the function of the structure. Some examples of anatomical anomalies include: Muscles (e.g., absence of muscles, doubled muscles, divided muscles, increased or decreased origin or insertion of the muscle, joining to adjacent organs); Bones (e.g., sometimes there are six lumbar vertebrae instead of the usual five); Joints (e.g., discoid meniscus, a rare thickened lateral meniscus in the knee joint); and Organ arrangement (e.g., 1 in 10000 people have their organs arranged in a mirror image (situs inversus)—the liver is on the left, the heart on the right, and so on). Other examples of anatomical anomalies include: Supernumerary bony structures; Missing or supernumerary muscle heads and bellies; Changes in the shape and position of organs and their parts; Supernumerary (accessory), missing, aberrant, and rudimentary vessels; and Differing numbers of lymph nodes.

Additionally, in certain illustrated embodiments, the aforesaid AI device 3000 and/or AI server 4000 may be used by server system 132 to determine the plurality of selectable anatomical anomalies that may be relevant to the patient via one or more AI/ML techniques, preferably based upon data relevant to the patient.

Once the user (e.g., a surgeon) has the proper view of the patient displayed on the GUI of device 116 (step S100) (and any anatomical anomalies relevant to the patient are selected), next at step S105, the user selects 6300, preferably via touch input on the GUI of device 116, one or more displayed human portions (6302) that are to be associated with one or a medical procedures (6304) performed on the patient. (FIG. 22D). Next, at step S110, the server system 132 determines, and causes to be displayed on the GUI of device 116, for user selection (6400), one or more medical/surgical procedures (6402-6406) that may be associated with the displayed 3D human body portion 6410 (FIG. 22E). Preferably, the one or more medical/surgical procedures (6402-6408) determined and displayed for user selection (6400) are contingent upon the one or more portions of the human body selected by the user (step S105), and user selected anatomical anomalies determined relevant to the patient. In certain illustrated embodiments, the aforesaid AI device 3000 and/or AI server 4000 may be used by system 132 to determine the one or more medical procedures (6402-6408) identified on the GUI of device 116 (as shown for example in FIG. 22D), via one or more AI/ML techniques, preferably based upon data relevant to the patient. Thus, and as described above, the computer system 132 need not rely upon prestored data and/or any templates for any aforesaid medical/surgical procedural determinations, which is again particularly advantageous. For instance, a surgeon has real-time access to up-to-date medical procedural information (which may be provided by other surgeons, on a worldwide basis) via access to one more relevant databases (e.g., memory 3700 and/or 2228), via communications network 1010.

Next, at step S115, the user selects (6500), on the GUI of device 116, one or more indicated medical procedures (6502-6506) that are displayed on the GUI of device 116 (step S110) that were actually performed on the patient with respect to the selected human body portions (FIG. 22E). In certain illustrated embodiments, the system 132 is further configured and operative to determine, and generate on the GUI of device 116, for user selection on the GUI of device 116: 1) one or more pharmaceuticals administered during the medical procedure for inclusion in the surgical report; 2) one or more pharmaceuticals prescribed for post-surgery treatment of the patient; and 3) one or more medical instrumentalities utilized during the medical procedure for inclusion in the surgical report. It is to be appreciated and understood that the aforesaid AI device 3000 and/or AI server 4000 may be used by system 132 to determine aforesaid pharmaceuticals and/or medical instruments via one or more AI/ML techniques, preferably based upon the selected medical procedures performed (step S115) and data relevant to the patient. Thus, the system 200 need not rely upon prestored data for these determinations and/or any templates, which is particularly advantageous.

Proceeding to step S120, the system 132 then preferably determines the billing codes associated with the selected medical procedures performed on the patent (step S115) as well as descriptive text associated with the performed medical procedures, step S125, so as to generate a medical/surgical report (FIG. 15) in accordance with the illustrated embodiments described herein, step S130. It is to be appreciated and understood that the aforesaid AI device 3000 and/or AI server 4000 may be used by system 200 to determine aforesaid the billing codes associated with the selected medical procedures performed on the patent (step S115) as well as descriptive text associated with the performed medical procedures, step S125. Thus, the system 132 need not rely upon prestored data for these determinations and/or any templates, which is again particularly advantageous but rather has access to current/up-to-date billing related data via access to one more relevant database, via communications network 1010.

Accordingly, the aforesaid generated medical/surgical report (FIG. 15) is preferably automatically generated based upon a user's input for producing an operative report for the selected surgical procedure(s) performed, as mentioned above. The report is then preferably transmitted, by system 132, via communications network 1010, to at least one destination for billing purposes (e.g., an insurance company) and/or a record storage repository (e.g., for retention purposes), and/or is processed/utilized for any other suitable purpose.

As mentioned above, the GUI of device 116 is preferably generated on a user touch interactive computer display device 116 wherein the GUI is preferably operated without resort to a keyboard or a pointing device. Preferably, in some embodiments, for security purposes (e.g., to secure patient medical data), the computer device 116 is preferably a “closed” device that is use restricted for generating a medical report as described herein, and thus provides no other functionality than as described herein.

As also mentioned above, in certain illustrated embodiments, the system 132 is provided with self-learning/Artificial Intelligence (AI) (e.g., AI system 3000 and AI server 400) for predicting and/or further automating one or more user selected surgical procedures/diagnosis' as mentioned above. For instance, when a surgical procedure is selected for a certain patient, based on historical data relating to such a selected surgical procedure (e.g., patient history, surgeon history, hospital history, geographic information, age, gender, and the like), AI system 3000 selects the predicted steps and procedures relating to the user selected procedure to be presented on the GUI (e.g., FIG. 22E). Additionally, the AI system 3000 may additionally be integrated into system 132 with an AI Surgical Planning system, that implements machine learning and artificial intelligence algorithms to identify recommendations for user presented parameters (via the GUI) for rapidly generating a surgical report.

The foregoing description has been directed to specific embodiments of the present invention. It will be apparent, however, that other variations and modifications may be made to the described embodiments, with the attainment of some or all of their advantages. Therefore, it is the object of the appended claims to cover all such variations and modifications as come within the true spirit and scope of the invention.

What is claimed is:

1. A computer-implemented method for generating a surgical report indicative of a medical procedure performed on a patient, comprising:
generating, on a computer display, a Graphical User Interface (GUI), the GUI including a user interactive animated visual representation of at least a portion of a human body representative of the patient;
receiving input from a user, on the GUI, wherein the input selects one or more portions of the human body visual representation that are associated with the medical procedure;
indicating, on the GUI, one or more medical procedures that may be associated with the medical procedure performed on the patient with respect to the selected human body portions;
receiving input from the user, on the GUI, wherein the input selects one or more indicated medical procedures performed on the patient with respect to the selected human body portions;
generating billing codes associated with the medical procedures that were performed;
generating text, based on input received via the GUI, identifying at least some of the medical procedures that were performed; and
generating a surgical report including the generated billing codes, and the generate text corresponding to the selected medical procedures performed on the patient associated with the surgical procedure.

2. The computer-implemented method as recited in claim 1, further including, generating for user selection on the GUI, a plurality of selectable anatomical anomalies that may be relevant to the patient, wherein the one or more portions of the human body, and the one or more medical procedures identified on the GUI, are contingent upon the one or more user selected anatomical anomalies determined relevant to the patient.

3. The computer-implemented method as recited in claim 2, wherein the plurality of selectable anatomy particulars that may be relevant to the patient include at least one of: 1) a position of an organ on the patient's human body; and 2) a stage of pregnancy associated with the patient.

4. The computer-implemented method as recited in claim 3, wherein one or more Artificial Intelligence (AI) techniques are utilized for determining at least one of the plurality of selectable anatomy particulars or the one or more medical procedures identified on the GUI.

5. The computer-implemented method as recited in claim 1, wherein the computer display is touch screen device and wherein the user input consists of detection of user touch on the GUI.

6. The computer-implemented method as recited in claim 1, wherein the computer display is touch screen device and wherein the user input consists of use of a stylus device.

7. The computer-implemented method as recited in claim 1, wherein the user input consists of dictation techniques.

8. The computer-implemented method as recited in claim 1, wherein the computer display is provide by a Virtual Reality (VR) headset.

9. The computer-implemented method as recited in claim 1, wherein the display device receives, via a communications network, instructions and data from a remotely located computer for enabling operation of the GUI, wherein the remotely located computer generates the surgical report.

10. The computer-implemented method as recited in claim 3, wherein the remotely located computer is a cloud-based computer system.

11. The computer-implemented method as recited in claim 11, wherein the GUI is provided by a portable user smart computing device.

12. The computer-implemented method as recited in claim 1, wherein the interactive animated visual representation of at least a portion of the human body representative of the patient is a three-dimensional (3D) model of the human body portion that has an adjustable view perspective on the GUI via manipulation of the 3D human body model caused by user interactive touch input upon the GUI.

13. The computer-implemented method as recited in claim 1, wherein the adjustable view perspective of the 3D human body portion displayed on the GUI, via user manipulation of the 3D human body model, causes, for user selection, one or more medical procedures to be displayed on the GUI associated with the displayed 3D human body portion.

14. The computer-implemented method as recited in claim 1, further including generating, on the GUI, a menu that is common to each display generated on GUI associated with the surgical procedure whereby the menu is interactive to highlight a portion of the menu associated with one or more of the medical procedures associate with the surgical procedure currently displayed on the GUI.

15. The computer-implemented method as recited in claim 1, further including, displaying for user selection on the GUI, one or more pharmaceuticals administered during the medical procedure for inclusion in the surgical report.

16. The computer-implemented method as recited in claim 1, further including, displaying for user selection on the GUI, one or more pharmaceuticals prescribed for post-surgery treatment of the patient.

17. The computer-implemented method as recited in claim 1, further including, displaying for user selection on the GUI, one or more medical instrumentalities utilized during the medical procedure for inclusion in the surgical report.

18. The computer-implemented method as recited in claim 1, further including, transmitting, via a communications network, the generated medical report to a designated recipient.

19. The computer-implemented method as recited in claim 18, wherein the designated recipient is an insurance entity.

20. The computer-implemented method as recited in claim 1, wherein one or more Artificial Intelligence (AI) techniques are utilized for generating the medical report wherein data captured for AI analysis for generating the medical report includes usage of a Large Language Model (LLM).

21. A computer system for generating a surgical report indicative of a medical procedure performed on a patient, comprising:
a memory;
a processor disposed in communication with said memory, and configured to issue a plurality of instructions stored in the memory, wherein the instructions cause the processor to:
generate, on a computer display, a Graphical User Interface (GUI), the GUI including a user interactive animated visual representation of at least a portion of a human body representative of the patient, further including generating for user selection on the GUI, a plurality of selectable anatomical anomalies that may be relevant to the patient, wherein the one or more portions of the human body, and the one or more medical procedures identified on the GUI, are contingent upon the one or more user selected anatomical anomalies determined relevant to the patient;
receive, input from a user, on the GUI, wherein the input selects one or more portions of the human body visual representation that are associated with the medical procedure and the one or more user selected anatomical anomalies determined relevant to the patient;
indicate, on the GUI, one or more medical procedures that may be associated with the medical procedure performed on the patient with respect to the selected human body portions;
receive, input from the user, on the GUI, wherein the input selects one or more indicated medical procedures performed on the patient with respect to the selected human body portions;
generate billing codes associated with the medical procedures that were performed;
generate text, based on input received via the GUI, identifying at least some of the medical procedures that were performed; and
generate a surgical report including the generated billing codes, and the generate text corresponding to the selected medical procedures performed on the patient associated with the surgical procedure.

22. The computer-implemented system as recited in claim 21, wherein one or more Artificial Intelligence (AI) techniques are utilized for determining at least one of the plurality of selectable anatomy particulars or the one or more medical procedures identified on the GUI and for generating the medical report wherein data captured for AI analysis for generating the medical report includes usage of a Large Language Model (LLM).

23. The computer-implemented system as recited in claim 21, wherein the computer system consists of a computer server remotely located from one or more user portable computing devices, wherein each user portable computing device includes the generated user interactive GUI.
